(19) United States
(12) Patent Application Publication
(10) Pub. No.: US 2004/0097911 A1
(43) Pub. Date: May 20, 2004

(54) Ultrasonic operating apparartus and tool for changing tip thereof

(75) Inventors: Eiji Murakami, Hachioji-shi (JP); Kenichi Kimura, Hachioji-shi (JP)

Correspondence Address: OSTROLENK FABER GERB & SOFFEN, 1180 AVENUE OF THE AMERICAS, NEW YORK, NY, 100368403

(73) Assignee: Olympus Optical Co., Ltd.

(21) Appl. No.: 10/614,620

(22) Filed: Jul. 7, 2003

Related U.S. Application Data
(63) Continuation-in-part of application No. 10/074,787, filed on Feb. 12, 2002, now abandoned.

(30) Foreign Application Priority Data
Feb. 13, 2001 (JP) 2001-035921
Jul. 4, 2002 (JP) 2002-196344
Jul. 19, 2002 (JP) 2002-211372

(51) Int. Cl. A61B 18/04

(52) U.S. Cl. 606/27

(57) ABSTRACT

According to the present invention, a jaw unit is provided with a frame-shaped jaw body and a tip for seizing an organism tissue in conjunction with an operating portion of a vibration transmitting member, and the tip is removably coupled between arms of the jaw body.

### CROSS-REFERENCE TO RELATED APPLICATIONS

This is a Continuation-in-Part application of U.S. patent application Ser. No. 10/074,787, filed Feb. 12, 2002, the entire contents of which are incorporated herein by reference.

This application is based upon and claims the benefit of priority from the prior Japanese Patent Applications No. 2002-196344, filed Jul. 4, 2002; and No. 2002-211372, filed Jul. 19, 2002, the entire contents of both of which are incorporated herein by reference.

### BACKGROUND OF THE INVENTION

1. Field of the Invention

The present invention relates to an ultrasonic operating apparatus capable of performing operation, such as incision, ablation, or coagulation of an organism tissue, by utilizing ultrasonic waves while seizing the organism tissue between an ultrasonic probe and a jaw, and a tool for changing a tip thereof.

2. Description of the Related Art

An apparatus described in Jpn. Pat. Appln. KOKAI Publication No. 10-5236 or the like is an example of an ultrasonic operating apparatus that performs operation, such as incision, ablation, or coagulation of an organism tissue, by utilizing ultrasonic waves, in general. In this ultrasonic operating apparatus, a handling portion on the hand side is coupled to the proximal end portion of an insert portion covering tube. This handling portion is provided with an ultrasonic vibrator that generates ultrasonic vibration. Further, an operating portion for operating the organism tissue is provided on the distal end portion of the insert portion covering tube.

The insert portion covering tube is penetrated by a vibration transmitting member that transmits the ultrasonic vibration from the ultrasonic vibrator to an ultrasonic probe on the operating portion side. The proximal end portion of the vibration transmitting member is connected to the ultrasonic vibrator. Further, the operating portion is provided with a jaw that is rockably supported opposite the ultrasonic probe. A tip of the jaw that touches the organism tissue is formed of a plastic material such as Teflon (trademark).

The operating portion is provided with a control handle for opening and closing the jaw with respect to ultrasonic probe. Further, a handling rod of the jaw is inserted in the insert portion covering tube for axial movement. As the control handle is operated, the handling rod is advanced or retreated in the axial direction. In association with this movement of the handling rod, the jaw of the operating portion is opened or closed with respect to the ultrasonic probe. As the jaw is opened or closed, the organism tissue can be seized between the ultrasonic probe and the jaw. Subsequently, in this state, the ultrasonic vibration from the ultrasonic vibrator is transmitted to the ultrasonic probe on the operating portion side by means of the vibration transmitting member. Thus, operation, such as incision, ablation, or coagulation of the organism tissue, can be performed by utilizing ultrasonic waves.

The ultrasonic operating apparatus is repeatedly used in a plurality of operations. During operation, the ultrasonic vibration of the ultrasonic probe is transmitted to the jaw of the operating portion. Thus, the tip of the plastic material used in the jaw of the operating portion is gradually worn away with the passage of time. All other components around the jaw of the operating portion than the tip are more durable than the tip of the jaw. If the tip of the jaw is worn away and rendered unusable, therefore, the components other than the tip can be kept usable.

In the conventional configuration described above, however, all the jaw components including the tip are integrally combined and unitized. If the tip of the jaw is worn away and rendered unusable, therefore, all the parts that are combined with the jaw and unitized must be replaced. Accordingly, the cost of parts replacement increases, so that it is hard to lower the running cost of the ultrasonic operating apparatus.

Further, the ultrasonic coagulotomy apparatus described in Jpn. Pat. Appln. KOKAI Publication No. 10-5236 is provided with a rotation drive mechanism for rotating the jaw of the operating portion around the central axis of the ultrasonic probe. If the distal end portion of the ultrasonic probe of the operating portion is curved rightward or leftward with respect to the direction of the center line, directivity develops according to the curved shape of the ultrasonic probe of the operating portion.

On the actual scene of ultrasonic operation, for example, the ultrasonic probe sometimes may be expected to be turned upward or downward in the visual field of an endoscope, depending on the region to be operated. In such a case, the insert portion is rotated around its axis to move the ultrasonic probe in a desired direction by rotating a rotary knob of the operating portion in a desired direction.

With the above-described configuration, however, the direction of the ultrasonic probe at the distal end portion may be reversed despite the rotation of the insert portion, in some cases, so that use of the probe is not easy. Conventionally, to solve this problem, two differently oriented operating devices, e.g., a leftward-curve operating device and a rightward-curve operating device are prepared as separate bodies. The operating devices of the two types are suitably alternatively used by replacement, depending on working conditions such as the place of the region to be operated. In this case, therefore, it is necessary to separately prepare similar operating devices of the two types, left and right, having respective distal operating portions differently oriented, meaning that the whole ultrasonic operating apparatus is very expensive.

### BRIEF SUMMARY OF THE INVENTION

The present invention has been contrived in consideration of these circumstances, and its object is to provide an ultrasonic operating apparatus, designed so that the cost of the whole system can be lowered and the direction of a distal operating portion can be easily changed at low cost, and a tool for changing a tip thereof.

In order to achieve the above object, according to the present invention, there is provided an ultrasonic operating apparatus, which comprises: an elongate insert portion capable of being inserted into a body cavity; an operating portion located on a distal end portion of the insert portion, the operating portion being used to operate an organism tissue; a handling portion coupled to a proximal end portion of the insert portion, the handling portion having therein an ultrasonic vibrator capable of generating ultrasonic vibration; a covering tube located around the insert portion; a vibration transmitting member passed through the covering tube, the vibration transmitting member having an ultrasonic probe on a side of the operating portion and capable of transmitting the ultrasonic vibration from the ultrasonic vibrator to the ultrasonic probe; a jaw rockably supported opposite the ultrasonic probe and capable of seizing the organism tissue in conjunction with the ultrasonic probe; a control handle located in the handling portion and capable of opening and closing the jaw with respect to the ultrasonic probe; and a handling force transmitting member coupling the jaw and the control handle, and capable of transmitting handling force from the control handle to the jaw, the jaw including a frame-shaped jaw body having at least supporting arms arranged individually on the opposite sides of a slot extending in an axial direction of the insert portion, a tip capable of seizing the organism tissue in conjunction with the ultrasonic probe, and a joint portion removably coupling the tip between the supporting arms of the jaw body.

According to the present invention, the tip is removably coupled between the supporting arms of the jaw body of the jaw so that the tip can be removed from between the supporting arms if it is worn away, and thereafter, a new tip is mounted between the supporting arms for replacement. Further, two types of tips, left and right, having their respective distal operating portions directed differently, are suitably alternatively mounted between the supporting arms for replacement, depending on conditions such as the place of the region to be operated. Even in the case where the distal operating portion has an asymmetric portion with respect to the central axis of the insert portion and displays directivity as it rotates around the axis of the insert portion, the direction of the distal operating portion can be easily changed at low cost.

In the ultrasonic operating apparatus according to claim 1 of the present invention, moreover, the ultrasonic probe has an asymmetric curved portion curved with respect to the central axis of the insert portion covering tube.

According to the present invention, the position of the distal operating portion is deviated from a center position in the visual field of an endoscope by means of the curved portion of the ultrasonic probe, so that the distal operating portion is easily visible in the visual field of the endoscope.

In the ultrasonic operating apparatus according to claim 2 of the present invention, furthermore, the curved portion is formed symmetrically with respect to the direction in which the jaw is opened or closed.

Since the curved portion of the ultrasonic probe is formed symmetrically with respect to the direction in which the jaw is opened or closed, according to the present invention, the distal operating portion can be easily turned in two directions, left and right, by means of one apparatus, so that the number of operating apparatuses to be assorted can be reduced and the cost can be lowered.

In the ultrasonic operating apparatus according to claim 1 of the present invention, moreover, the jaw body is designed so that support shaft portions of the tip protrude inward from the respective distal end portions of the two supporting arms, and the tip has mounting holes into which the support shaft portions are removably inserted and guide grooves for guiding the support shaft portions to the mounting holes as the tip is attached to the jaw body, the guide grooves individually having taper surfaces for movement such that the space between the respective support shaft portions of the two supporting arms widens toward the mounting holes and click step portions for preventing the support shaft portions from slipping out of the mounting holes.

In attaching the tip to the jaw body, according to the present invention, the respective support shaft portions of the two supporting arms are guided along the guide grooves of the tip to the mounting holes. As the support shaft portions are moved along the guide grooves of the tip, they are moved in a direction such that the space between the respective support shaft portions of the two supporting arms widens toward the mounting holes. Then, the support shaft portions pass over the click steps at the junctions with the mounting holes and are inserted into the mounting holes of the tip. When the support shaft portions are coupled to the mounting holes of the tip, moreover, the click steps serve to prevent them from slipping out of the mounting holes.

A tool for changing a tip of an ultrasonic operating apparatus according to the present invention comprises: a tip changing tool body having an insertion hole into which a distal operating portion of the ultrasonic operating apparatus can be inserted and a stopper portion for locating the position of insertion of the distal operating portion inserted in the insertion hole; a handling arm coupled to the jig body so as to be rockable around a hinge portion located on the inlet side of the insertion hole of the jig body; and wedge-shaped separating portions adapted to be removably inserted into spaces between a tip for seizing an organism tissue and supporting arms on the opposite sides of a jaw body of the distal operating portion as the handling arm rocks, thereby moving the supporting arms in a direction such that indented fitting portions of the supporting arms and the tip are disengaged from one another.

In removing the tip from the jaw body, according to the present invention, the position of insertion of the distal operating portion of the ultrasonic operating apparatus is located by means of the stopper portion with the distal operating portion inserted in the insertion hole of the tip changing tool body. In this state, the handling arm is rocked around the hinge portion on the inlet side of the insertion hole of the jaw body with respect to the jig body. As the handling arm is rocked in this manner, the wedge-shaped separating portions are inserted into the spaces between the tip for seizing the organism tissue and the supporting arms on the opposite sides of the jaw body of the distal operating portion, whereby the supporting arms are moved in a direction such that the indented fitting portions of the supporting arms and the tip are disengaged from one another. By doing this, the tip is removed from the jaw body.

Additional objects and advantages of the invention will be set forth in the description which follows, and in part will be obvious from the description, or may be learned by practice of the invention. The objects and advantages of the invention may be realized and obtained by means of the instrumentalities and combinations particularly pointed out hereinafter.

### BRIEF DESCRIPTION OF THE SEVERAL VIEWS OF THE DRAWING

The accompanying drawings, which are incorporated in and constitute a part of the specification, illustrate presently preferred embodiments of the invention, and together with the general description given above and the detailed description of the embodiments given below, serve to explain the principles of the invention.

FIG. 1 is a side view showing an assembled state of the whole body of an ultrasonic operating apparatus of a first embodiment of the present embodiment;

FIG. 2 is a longitudinal sectional view showing the internal configuration of a handling portion in the ultrasonic operating apparatus of the first embodiment;

FIG. 3 is a sectional view taken along line III-III of FIG. 2;

FIG. 4 is a sectional view taken along line IV-IV of FIG. 2;

FIG. 5A is a side view showing a probe unit of the ultrasonic operating apparatus of the first embodiment;

FIG. 5B is a sectional view taken along line 5B-5B of FIG. 5A;

FIG. 5C is a sectional view taken along line 5C-5C of FIG. 5A;

FIG. 5D is a sectional view taken along line 5D-5D of FIG. 5A;

FIG. 5E is a sectional view taken along line 5E-5E of FIG. 5A;

FIG. 6 is an exploded perspective view of the distal end portion of an insert portion of a handle unit in the ultrasonic operating apparatus of the first embodiment;

FIG. 7 longitudinal sectional view showing a detailed configuration of a distal operating portion of the ultrasonic operating apparatus of the first embodiment;

FIG. 8A is a sectional view taken along line 8A-8A of FIG. 7;

FIG. 8B is a sectional view taken along line 8B-8B of FIG. 7;

FIG. 8C is a sectional view taken along line 8C-8C of FIG. 7;

FIG. 8D is a sectional view taken along line 8D-8D of FIG. 7;

FIG. 9 is a plan view showing a curved state of a jaw unit in the ultrasonic operating apparatus of the first embodiment;

FIG. 10 is a side view showing a closed state of the jaw unit in the ultrasonic operating apparatus of the first embodiment;

FIG. 11A is a plan view showing a curved portion of an operating portion of the probe unit in the ultrasonic operating apparatus of the first embodiment;

FIG. 11B is a sectional view taken along line 11B-11B of FIG. 11A;

FIG. 11C is a side view showing the curved portion of the operating portion;

FIG. 12 is a longitudinal sectional view of a principal part showing the internal configuration of portions surrounding a rotary knob in the ultrasonic operating apparatus of the first embodiment;

FIG. 13A is a plan view showing a distal operating portion of an ultrasonic operating apparatus according to a second embodiment of the present invention;

FIG. 13B is a side view of the distal operating portion;

FIG. 14A is a front view of a rightward-curve ultrasonic probe in the ultrasonic operating apparatus of the second embodiment;

FIG. 14B is a side view of the rightward-curve ultrasonic probe;

FIG. 14C is a front view of a leftward-Curve ultrasonic probe;

FIG. 14D is a side view of the leftward-curve ultrasonic probe;

FIG. 15 is a side view of a jaw unit in the ultrasonic operating apparatus of the second embodiment;

FIG. 16 is a sectional view taken along line 16-16 of FIG. 15;

FIG. 17 is a plan view, partially in section, showing a jaw body in the ultrasonic operating apparatus of the second embodiment;

FIG. 18A is a plan view showing a tip changing tool in the ultrasonic operating apparatus of the second embodiment;

FIG. 18B is a side view of a jig body;

FIG. 19A is a plan view showing a state in which the operating portion of the ultrasonic operating apparatus of the second embodiment is inserted in the tip changing tool of the ultrasonic operating apparatus;

FIG. 19B is a side view showing the same state;

FIG. 20A is a sectional view taken along line 20A-20A of FIG. 19B;

FIG. 20B is a longitudinal sectional view of a principal part for illustrating operation for combining the jaw body and a tip of the second embodiment;

FIG. 21 is a side view of an entire ultrasonic operating apparatus according to a third embodiment of the present invention;

FIG. 22A is a plan view showing a state in which a distal end portion of the ultrasonic operating apparatus according to the third embodiment is seen from the top;

FIG. 22B is a side view showing a state in which the distal end portion of the ultrasonic operating apparatus according to the third embodiment is seen from the side;

FIG. 23 is a longitudinal cross section of the distal end portion in the ultrasonic operating apparatus according to the third embodiment;

FIG. 24 is a sectional view taken along the line 24-24 in FIG. 23;

FIG. 25 is a longitudinal cross sectional view showing an internal construction of a handling portion in the ultrasonic operating apparatus according to the third embodiment;

FIG. 26 is a sectional view of the handling portion taken along the line 26-26 in FIG. 25;

FIG. 27A is a side view showing a probe of the ultrasonic operating apparatus according to the third embodiment;

FIG. 27B is a sectional view of the probe taken along the line 27B-27B in FIG. 27A;

FIG. 28 is a plan view showing a state in which a disassembling jig is mounted on a distal end portion of the ultrasonic operating apparatus according to the third embodiment;

FIG. 29A is a longitudinal cross sectional view showing a state before a seizing member of the ultrasonic operating apparatus according to the third embodiment is removed by using the disassembling jig;

FIG. 29B is a longitudinal cross sectional view showing a state of disassembling work when the seizing member is removed by using the above disassembling jig;

FIG. 30A is a plan view showing a state in which the disassembling jig is mounted at a distal end portion formed in a curve-type distal end shape in the ultrasonic operating apparatus according to the third embodiment;

FIG. 30B is a longitudinal cross sectional view showing a state in which the disassembling jig is mounted at a distal end portion formed in a curve-type distal end shape in the ultrasonic operating apparatus according to the third embodiment;

FIG. 31 is a side view showing a state in which a ultrasonic operating apparatus according to a fourth embodiment of the present invention is disassembled;

FIG. 32 is a side view showing a state in which components of the ultrasonic operating apparatus according to the fourth embodiment are assembled;

FIG. 33A is a longitudinal cross sectional view of essential portions showing a construction of a distal end portion of the ultrasonic operating apparatus according to the fourth embodiment; FIG. 33B is a cross sectional view taken along the line 33B-33B in FIG. 33A;

FIG. 33C is a side view showing a fragmentary cross section of a coupling portion between a vibration transmitting member of a probe unit and a handle unit;

FIG. 34 is a side view showing a fragmentary cross section of a rear side portion of the handle unit in the ultrasonic operating apparatus according to the fourth embodiment;

FIG. 35A is a side view of a first handling portion unit replacing portion in the ultrasonic operating apparatus according to the fourth embodiment;

FIG. 35B is a top view of the handling portion unit replacing member;

FIG. 35C is a side view of a distal end portion of a first probe unit replacing member;

FIG. 35D is a tip view of a distal end portion of a first probe unit replacing member;

FIG. 35E is a sectional view taken along the line 35E-35E in FIG. 35C;

FIG. 36A is a side view of a second handling portion unit replacing member in the ultrasonic operating apparatus according to the fourth embodiment;

FIG. 36B is a top view of a second handling portion unit replacing member;

FIG. 36C is a side view of a distal end portion of a second probe unit replacing member;

FIG. 36D is a tip view of a distal end portion of a second probe unit replacing member;

FIG. 37A is a side view showing a handle unit of a ultrasonic operating apparatus according to a fifth embodiment of the present invention;

FIG. 37B is a side view showing a plurality of probe unit replacing members;

FIG. 37C is a side view showing a vibrator unit;

FIG. 37D is a side view showing a plurality of handling portion unit replacing member; and

FIG. 38 is a side view showing a fragmentary cross section of a distal end portion of an insert portion in the ultrasonic operating apparatus according to the fifth embodiment.

### DETAILED DESCRIPTION OF THE INVENTION

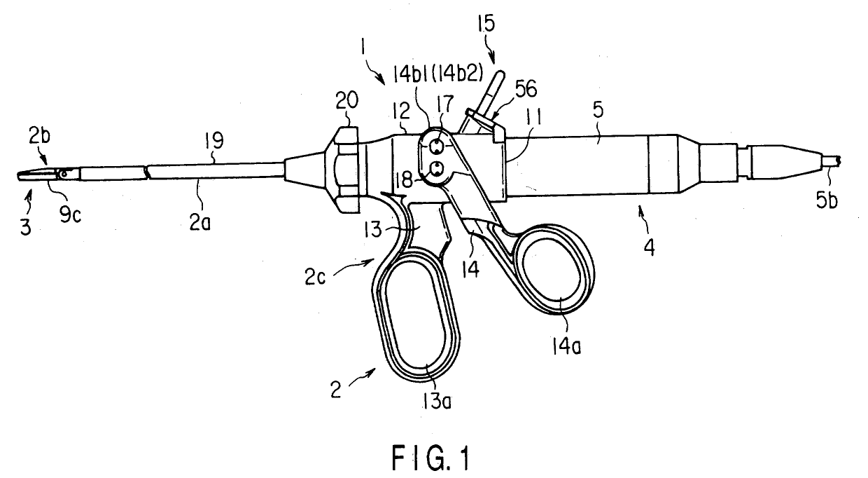

A first embodiment of the present invention will now be described with reference to FIGS. 1 to 12. FIG. 1 shows an assembled state of the whole body of an ultrasonic operating apparatus 1 of the present embodiment. This ultrasonic operating apparatus 1 comprises three assembly units that can be disassembled into three units, that is, a handle unit (handling portion) 2, a probe unit 3, and a vibrator unit 4. These three units 2 to 4 can be assembled into the state shown in FIG. 1.

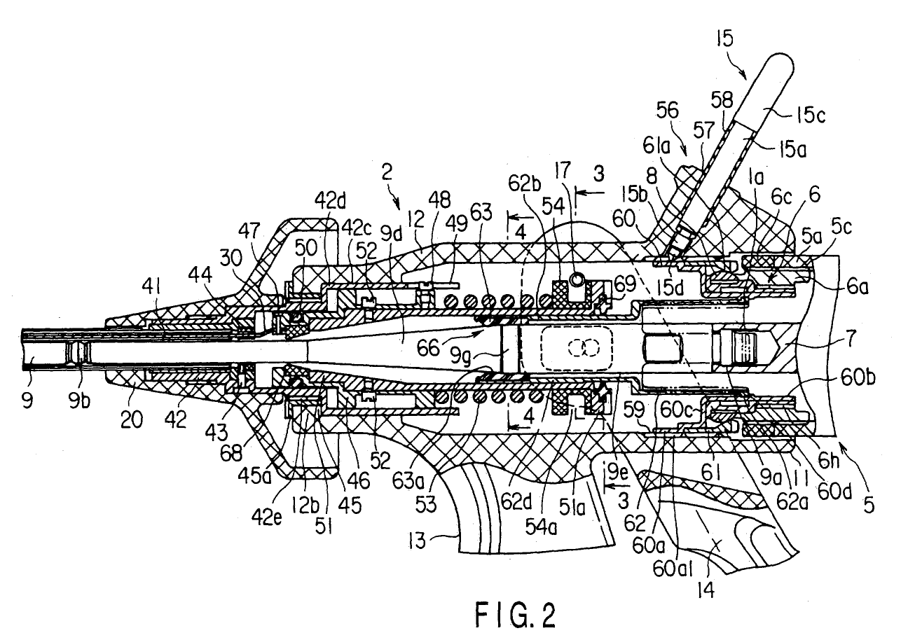

As shown in FIG. 2, the vibrator unit 4 has therein an ultrasonic vibrator (not shown) that generates ultrasonic vibration in a cylindrical vibrator cover 5a. Further, the proximal end portion of a horn 7 for enlarging the amplitude of ultrasonic vibration is coupled to the distal end portion of the ultrasonic vibrator. The distal end portion of the horn 7 is formed having a probe mounting tapped hole portion 7a.

Further, one end portion of a hand piece cord 5b for supplying current from a power source body (not shown) is connected to the rear end portion of the vibrator cover 5a. A hand piece plug (not shown) for connection to the power source body is connected to the other end portion of the hand piece cord 5b.

As shown in FIG. 2, a unit joint portion 6 for attachment and detachment of the handle unit 2 is attached to the distal end portion of the vibrator unit 4. The unit joint portion 6 is provided with a connecting ring 6a, ring-shaped attachment member 6b, fixing ring 6c, and engaging ring 8. An attachment mounting tapped hole portion 5c is formed in the inner peripheral surface of the distal end portion of the vibrator cover 5a. An external thread portion on the outer peripheral surface of the connecting ring 6a is screwed in the tapped hole portion 5c. Further, the fixing ring 6c is screwed on the distal end portion of the external thread portion of the connecting ring 6a.

Further, the outer peripheral surface of the proximal end portion of the attachment member 6b is screwed in the inner peripheral surface of the connecting ring 6a. The engaging ring 8 is fitted on the outer peripheral surface of the distal end portion of the attachment member 6b. The engaging ring 8 is formed of a so-called C-ring having the shape of a C obtained by cutting off a part of a ring. As shown in FIG. 2, the sectional shape of the engaging ring 8 is a substantially semilunar sectional shape such that its outer periphery is in the shape of a circular arc. This unit joint portion 6 can be detachably coupled to a vibrator connecting portion 11 of a handling portion body 12 (mentioned later) of the handle unit 2.

As shown in FIG. 5A, moreover, the probe unit 3 is provided with a vibration transmitting member 9 substantially in the form of an elongate rod that is detachably coupled to the tapped hole portion 7a on the distal end side of the horn 7 of the vibrator unit 4. The proximal end portion of the vibration transmitting member 9 is formed having a mounting screw 9a that is coupled to the tapped hole portion 7a of the horn 7. The mounting screw 9a is fixed to the tapped hole portion 7a of the horn 7 of the vibrator unit 4 by screwing. Thus, the probe unit 3 and the vibrator unit 4 are united together.

Further, rubber rings 9b, flange-shaped supports formed of a ring-shaped elastic member each, are provided individually in positions (a plurality of spots) for nodes of ultrasonic vibration that is transmitted from the side of the probe unit 3.

Further, an operating portion (ultrasonic probe) 9c is provided on the extreme distal end portion of the vibration transmitting member 9 of the present embodiment. As shown in FIG. 11A, the ultrasonic probe 9c is formed having a curved portion 10 in an asymmetric shape, e.g., the shape of a circular arc, which is curved away from a central axis 01, as shown in FIG. 11A.

As shown in FIG. 1, moreover, the handle unit 2 is composed of an elongate insert sheath portion 2a, a distal working portion 2b on the distal end portion of the insert sheath portion 2a, and a handing portion 2c on the proximal end portion of the insert sheath portion 2a. The handing portion 2c of the handle unit 2 is provided with the handling portion body 12 that is substantially cylindrical. The vibrator connecting portion 11 is formed on the proximal end portion of the handling portion body 12.

Further, a stationary handle 13 and a movable handle (handling means) 14 capable of rocking motion are provided on the outer peripheral surface of the handling portion body 12. Furthermore, an electrode pin 15 for high-frequency connection is attached to the top of the proximal end portion of the handling portion body 12 in a manner such that it is inclined backward.

The upper part of the stationary handle 13 is molded integrally with the cylindrical handling portion body 12. Further, the handling end portion of the stationary handle 13 is provided with a finger ring 13a in which a plurality of fingers other than the thumb can be selectively inserted, and the handling end portion of the movable handle 14 is provided with a finger ring 14a on which the thumb of the same hand can be hooked.

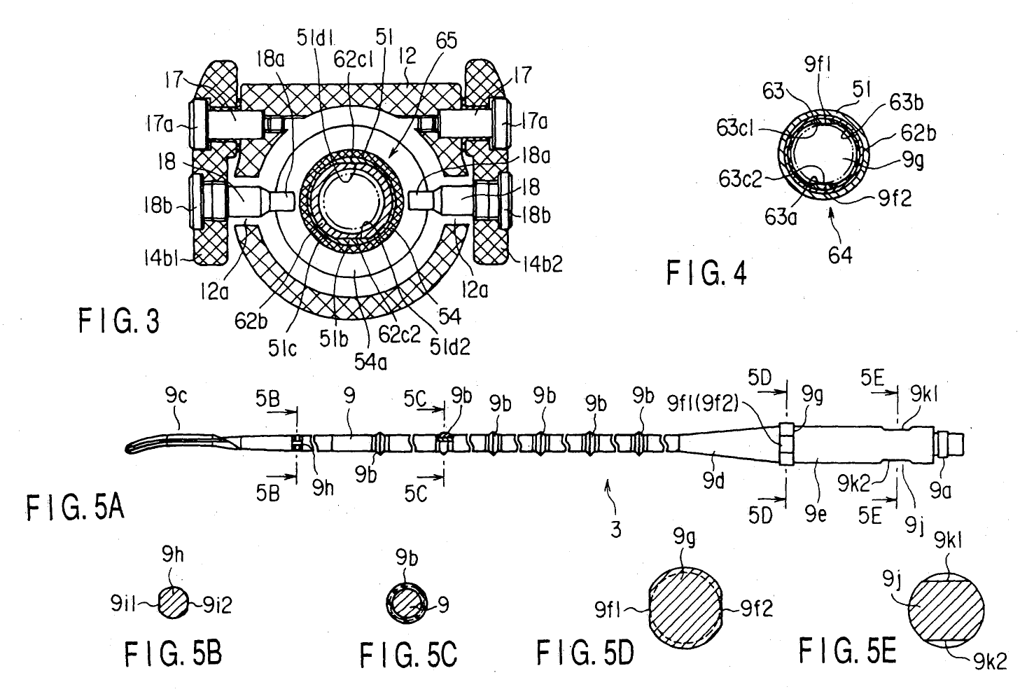

Bifurcate joint portions 14b1 and 14b2 are formed on the upper end side of the movable handle 14. As shown in FIG. 3, these bifurcate joint portions 14b1 and 14b2 are located individually on the opposite sides of the handling portion body 12. Further, the handle pivots 17 protrude inward from the respective upper end portions of the joint portions 14b1 and 14b2, individually. These handle pivots 17 are coupled to the handling portion body 12 at pivotal points above the axis of an insert portion covering tube 19 (mentioned later). Thus, the movable handle 14 is rockably supported by means of the handle pivots 17. The left- and right-hand handle pivots 17 are separately mounted so as not to project into the handling portion body 12. An insulating cap 17a for high-frequency insulation is attached to each handle pivot 17.

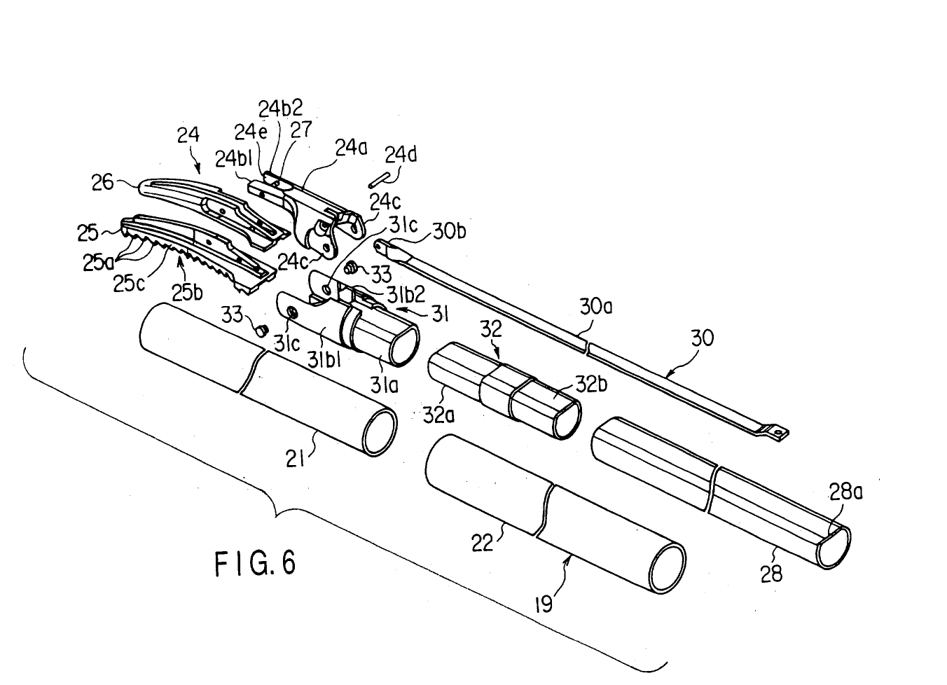

Further, actuator pins 18 for transmitting moving force to a handling rod (handling force transmitting member) 30 (mentioned later, see FIG. 6) project individually inward from the joint portions 14b1 and 14b2 of the movable handle 14 in regions near the handle pivots 17. These actuator pins 18 are located substantially on the axis of the insert portion covering tube 19. Windows 12a for the insertion of the actuator pins 18 are formed in the handling portion body 12. The actuator pins 18 of the movable handle 14 extend into the handling portion body 12 through the windows 12a of the handling portion body 12.

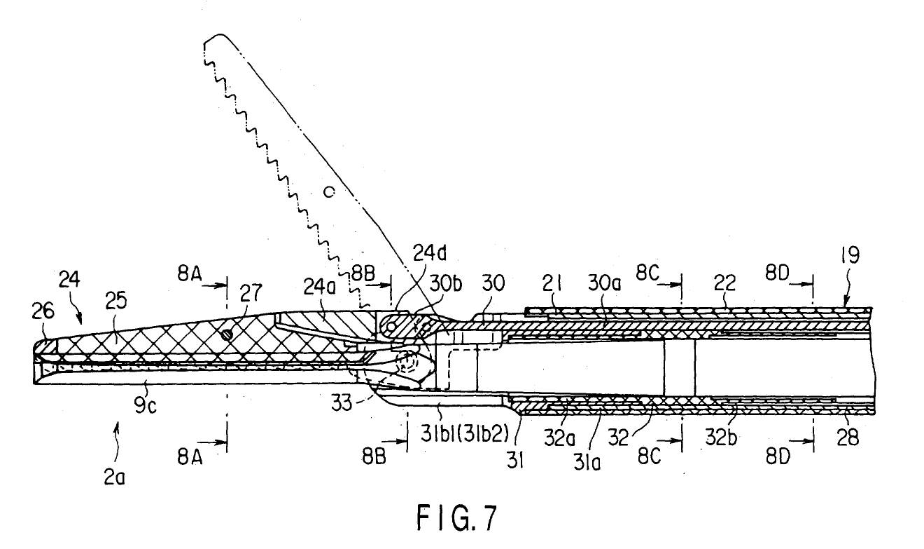

Furthermore, the insert sheath portion 2a is provided with the insert portion covering tube 19. The proximal end portion of the insert portion covering tube 19, along with a rotary knob (rotation drive mechanism) 20, is mounted on the distal end portion of the handling portion body 12 for rotation around the central axis of the insert portion covering tube 19. As shown in FIG. 7, the insert portion covering tube 19 is formed by fitting an insulating tube 22 on the outer peripheral surface of an outer pipe 21 that is formed of a metallic pipe. The insulating tube 22 is provided on the whole outer peripheral surface of the insert portion covering tube 19 so as to cover the greater part that reaches the proximal end portion.

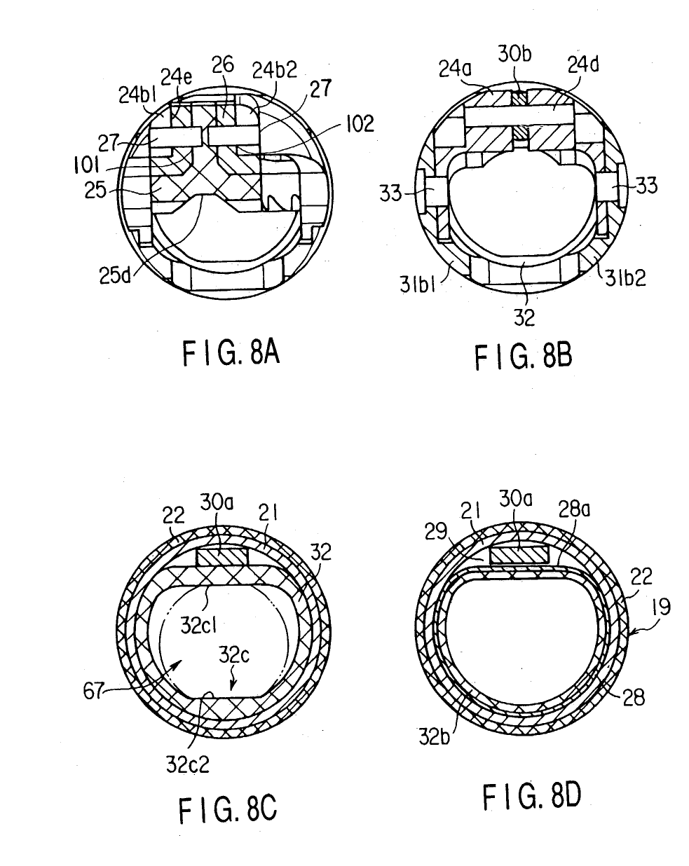

Further, a single-swing jaw unit 24 for seizing an organism tissue is rotatably attached to the distal working portion 2b of the handle unit 2. As shown in FIGS. 6 and 8B, the jaw unit 24 is provided with a substantially U-shaped jaw body 24a, a tip 25 for seizing an object (organ), and a seizing portion mounting member 26.

Furthermore, leg portions 24c that are bent diagonally backward, as shown in FIG. 6, are formed individually on the respective proximal end portions of a pair of U-shaped arms (supporting arms) 24b1 and 24b2 of the jaw body 24a.

As shown in FIG. 8A, moreover, the respective outer end portions of supporting pins (support shaft portions) 27 for supporting the tip 25 are fixed individually to the respective distal end portions of the arms 24b1 and 24b2 of the jaw body 24a. The supporting pins 27 project inside the arms 24b1 and 24b2, individually. As shown in FIG. 8B, moreover, a coupling pin 24d for connection with a handling rod 30 (mentioned later) is inserted in the respective upper edge portions of the leg portions 24c of the jaw body 24a.

The tip 25 is attached to a slit 24e between the arms 24b1 and 24b2 of the jaw body 24a by means of the seizing portion mounting member 26. The tip 25 is formed of a low-friction material such as PTFE (Teflon: trademark).

As shown in FIG. 8A, moreover, the tip 25 and the seizing portion mounting member 26 are formed having insertion holes 101 and 102, respectively, for the supporting pins 27. In assembling the jaw unit 24, the supporting pins 27 of the jaw body 24a are removably inserted into insertion holes 101 and 102 of the tip 25 and the seizing portion mounting member 26 and are removably coupled thereto. Thus, the tip 25 and the seizing portion mounting member 26 are swingably supported on the jaw body 24a by means of the supporting pins 27. When the tip 25 of the jaw unit 24 is pressed against the operating portion 9c of the vibration transmitting member 9 as the jaw unit 24 is closed, the tip 25 of the jaw unit 24 is caused to swing around the supporting pins 27, following the deflection of the operating portion 9c, so that the object (organ) can be seized with a uniform force by means of the whole contact portion between the tip 25 and the operating portion 9c.

Further, a plurality of nonskid teeth 25a are arranged on a contact surface of the tip 25 that touches the organism tissue as an object of coagulotomy, whereby a serrated nonslip tooth portion 25b is formed. The organism tissue as the object of coagulotomy can be seized without a slip by means of the nonskid tooth portion 25b of the tip 25.

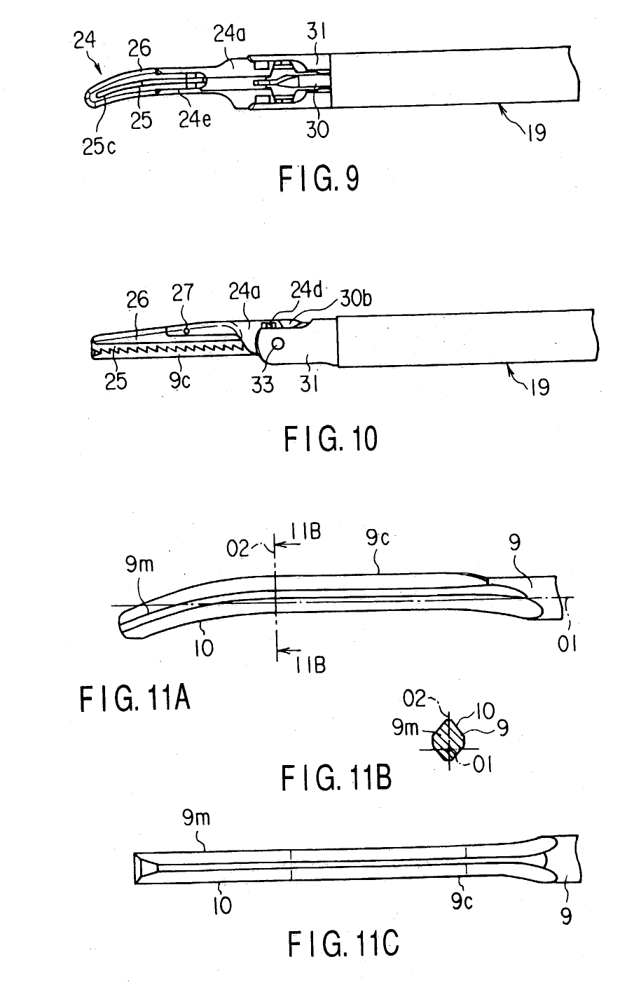

As shown in FIGS. 6 and 9, a curved portion 25c in the shape of a circular arc corresponding to the curved portion 10 of the vibration transmitting member 9 is formed on that surface of the tip 25 of the jaw unit 24 of the present embodiment which is opposed to the operating portion 9c of the vibration transmitting member 9. As shown in FIG. 8A, moreover, a seizing surface 25d in the shape of a recess corresponding to the shape (see FIG. 11B) of a contact surface 9m of the operating portion 9c of the vibration transmitting member 9 is formed on that surface of the tip 25 which is opposed to the operating portion 9c. When the jaw unit 24 is in its fully-closed position, the seizing surface 25d on the underside of the tip 25 is intimately in contact with the contact surface 9m of the operating portion 9c of the vibration transmitting member 9 without a gap.

An inner pipe 28 for use as a channel pipe is passed through the interior of the insert portion covering tube 19. As shown in FIGS. 6 and 8D, the inner pipe 28 has a substantially D-shaped cross section that includes a flat portion 28a formed in a part of a circular outer peripheral surface. The vibration transmitting member 9 of the probe unit 3 is passed through the inner pipe 28. Further, a sub-channel 29, a crescent space, is formed between the insert portion covering tube 19 and the flat portion 28a of the inner pipe 28. The handling rod 30 that transmits handling force for opening and closing the jaw unit 24 is movably passed through the sub-channel 29.

As shown in FIG. 6, this handling rod 30 has a rod body 30a that is formed of a substantially level platelike member. Further, the distal end portion of the handling rod 30 is formed having an upright jaw joint portion 30b that is obtained by twisting the flat rod body 30a at about 90°. The jaw joint portion 30b and the respective upper edge portions of the leg portions 24c are rockably coupled by means of the coupling pin 24d.

A jaw holding member 31 for holding the jaw unit 24 is attached to the distal end portion of the insert portion covering tube 19. As shown in FIG. 6, a substantially tubular fit-fixing portion 31a is formed on the proximal end portion of the jaw holding member 31. The fit-fixing portion 31a of the jaw holding member 31 is fixed by fitting to a distal end portion 32a of the coupling pipe 32 that is located in the insert portion covering tube 19. Further, the distal end portion of the inner pipe 28 is coupled to a proximal end portion 32b of the coupling pipe 32.

As shown in FIG. 8B, moreover, a pair of arm-shaped jaw mounting portions 31b1 and 31b2, left and right, are formed on the distal end portion of the jaw holding member 31. Further, pivot holes 31c are formed in the jaw mounting portions 31b1 and 31b2, individually. Pivot pins 33 that serve as pivots of the jaw body 24a are fitted individually in the respective pivot holes 31c of the jaw mounting portions 31b1 and 31b2. The jaw body 24a is mounted on the jaw holding member 31 for rotation around the pivot pins 33 as pivots. Thus, the jaw unit 24 can be opened or closed as the handling rod 30 is moved in the axial direction. The jaw unit 24 is closed when the handling rod 30 is pushed toward the distal end. In closing the jaw unit 24, the tip 25 of the jaw unit 24 is pressed against the operating portion 9c of the vibration transmitting member 9 of the probe unit 3, whereby the object (organ) can be seized between the operating portion 9c and the tip 25 of the jaw unit 24. The jaw unit 24 is also used to separate the organism tissue.

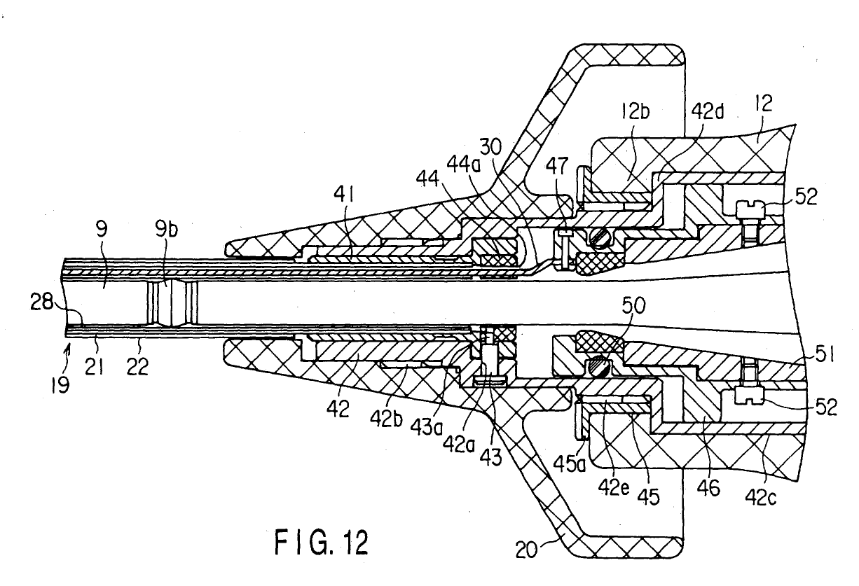

As shown in FIG. 12, a pipe fixing member 41 is fixed to the outer peripheral surface of the proximal end portion of the outer pipe 21 of the insert portion covering tube 19. A substantially cylindrical eccentric barrel 42 is mounted on the outer peripheral surface of the pipe fixing member 41. The center line of the eccentric barrel 42 is eccentric to the center line of the insert portion covering tube 19.

Further, a pit portion 42a is bored radially in the proximal end portion of eccentric barrel 42. A guide pin 43 is inserted in the pit portion 42a. The distal end portion of the guide pin 43 is fitted in the proximal end portion of the pipe fixing member 41.

Furthermore, a retaining ring 44 of a plastic material is fitted on the proximal end portion of the pipe fixing member 41. The inner peripheral surface of the retaining ring 44 has a diameter smaller than the inside diameter of the inner pipe 28. Thus, the metallic inner pipe 28 can be prevented from directly touching the vibration transmitting member 9. A handling rod passage hole 44a is formed in the retaining ring 44. The proximal end portion of the handling rod 30 is passed through the passage hole 44a.

Further, the retaining ring 44 is fitted with a small-diameter distal end protrusion 43a that protrudes from the distal end portion of the guide pin 43. Thus, the respective rotational-direction positions of the outer pipe 21 of the insert portion covering tube 19, pipe fixing member 41, eccentric barrel 42, and retaining ring 44 are regulated by means of the guide pin 43.

Furthermore, a rotary knob mounting screw portion 42b in the form of an external thread is formed on the outer peripheral surface of the eccentric barrel 42. This rotary knob mounting screw portion 42b is mated with an internal thread portion formed on the inner peripheral surface of the rotary knob 20 and is fitted with the rotary knob 20. Thus, as the rotary knob 20 rotates, the turning force of the rotary knob 20 is transmitted to the guide pin 43, pipe fixing member 41, retaining ring 44, the outer pipe 21 of the insert portion covering tube 19, and inner pipe 28, as well as to the eccentric barrel 42, whereupon these elements are rotated integrally with the rotary knob 20.

As shown in FIG. 2, moreover, a large-diameter rotating barrel portion 42c that extends to the interior of the handling portion body 12 is located on the proximal end side of the eccentric barrel 42. FIG. 2 shows the internal configuration of the handle unit 2. An inwardly bent flange portion 12b protrudes from the front end portion of the handling portion body 12.

Further, a substantially cylindrical rotating barrel portion 42c is fitted into the distal end opening of the handling portion body 12 from behind. As shown in FIG. 3, the rotating barrel portion 42c is formed having a first external thread portion 42e that has an inside diameter smaller than that of the flange portion 12b of the handling portion body 12 and is situated ahead of a shoulder portion 42d in engagement with the inner surface of the flange portion 12b.

Furthermore, a fixing ring 45 is screwed from the front side into the space between the flange portion 12b and the first external thread portion 42e of the rotating barrel portion 42c that is inserted in the handling portion body 12. The fixing ring 45 is in mesh with the first external thread portion 42e of the rotating barrel portion 42c. The flange portion 12b on the front end of the handling portion body 12 is held between a flange portion 45a on the distal end of the fixing ring 45 and the shoulder portion 42d of the rotating barrel portion 42c.

When the insertion end portion of the fixing ring 45 is in engagement with the shoulder portion 42d of the rotating barrel portion 42c, the distance between the shoulder portion 42d of the rotating barrel portion 42c and the proximal-end-side end face of the flange portion 45a of the fixing ring 45 is a little greater than the axial direction of the flange portion 12b. Thus, the rotating barrel portion 42c and the fixing ring 45 can be integrally rotated with respect to the flange portion 12b. The eccentric barrel 42 that has the diameter smaller than that of the first external thread portion 42e is coupled to the distal end portion of the rotating barrel portion 42c.

Further, a drive shaft connecting member (advancing/retreating member) 46 is inserted in the rotating barrel portion 42c for movement along the center line of the insert portion covering tube 19. The proximal end portion of the handling rod 30 is fixed to the distal end portion of the drive shaft connecting member 46 by means of a fixing pin 47.

Furthermore, a rotary fixing pin 48 protrudes from the proximal end portion of the drive shaft connecting member 46. The outer end portion of the rotary fixing pin 48 is inserted in a slot-shaped engaging groove 49 that is formed in the proximal end portion of the rotating barrel portion 42c. The engaging groove 49 extends in the axial direction of the insert portion covering tube 19. The rotating barrel portion 42c and the drive shaft connecting member 46 are relatively movable in the direction and are prevented from moving relatively to each other in the rotating direction by the rotary fixing pin 48.

When the rotary knob 20 is rotated, therefore, the force to rotate the rotary knob 20 is transmitted from the rotating barrel portion 42c rotating integrally with the eccentric barrel 42 to the drive shaft connecting member 46 via the rotary fixing pin 48. Thus, members that include the insert portion covering tube 19 and the members therein, the eccentric barrel 42 and the rotating barrel portion 42c mounted on the proximal end portion of the insert portion covering tube 19, and the rotary knob 20 can rotate integrally with the drive shaft connecting member 46 with respect to the handling portion body 12.

Further, an O-ring 50 is fitted on the outer peripheral surface of the drive shaft connecting member 46. The O-ring 50 serves to maintain airtightness between the rotating barrel portion 42c and the outer peripheral surface of the drive shaft connecting member 46.

Furthermore, the distal end portion of a slider mounting member 51 is screwed to the inner peripheral surface of the drive shaft connecting member 46 by means of fixing screws 52. An outward flange portion 51a that is bent outward protrudes from the proximal end portion of the slider mounting member 51.

Further, a limiting spring 53 formed of a coil spring and a ring-shaped slider 54 for spring bearing are arranged on the outer peripheral surface of the slider mounting member 51. The limiting spring 53 is mounted between the drive shaft connecting member 46 and the slider 54. The limiting spring 53 is compressed to be shorter than its free length and subjected to an equipment load as it is set in position.

Furthermore, a ring-shaped engaging groove 54a that engages the movable handle 14 is formed on the outer peripheral surface of the slider 54. As shown in FIG. 3, the respective inner end portions of the actuator pins 18 of the joint portions 14b1 and 14b2 of the movable handle 14 are inserted into the engaging groove 54a through the windows 12a of the handling portion body 12, individually. Small-diameter distal end engaging portions 18a corresponding in size to the groove width of the engaging groove 54a of the slider 54 are formed individually on the respective inner end portions of the actuator pins 18. The distal end engaging portions 18a of the actuator pins 18 are inserted into the engaging groove 54a of the slider 54 and engage the engaging groove 54a so as to be slidable in the circumferential direction along it. The actuator pins 18 are screwed to the joint portions 14b1 and 14b2 of the movable handle 14, individually. Further, an insulating cap 18b for high-frequency insulation is attached to the outer end portion of each actuator pin 18.

When the movable handle 14 is gripped (closing operation), the actuator pins 18 are rotated in the clockwise direction of FIG. 1 around the handle pivots 17. As this is done, the actuator pins 18 are advanced substantially straight to the distal end side within the ranges of movement of the actuator pins 18. This motion of the actuator pins 18 causes the slider 54 to advance toward the distal end. Further, this advancing motion of the slider 54 is transmitted from the slider mounting member 51 to the drive shaft connecting member 46 by means of the fixing screws 52, and the handling rod 30 is pushed out toward the distal end by means of the drive shaft connecting member 46. Since the limiting spring 53 is compressed to be shorter than its free length and subjected to the equipment load as it is mounted, it can directly cause the jaw unit 24 to open or close without undergoing elastic deformation with a handle operating force lighter than the equipment load, thereby improving the handling. If a force heavier than the equipment load of the limiting spring 53 is applied, the limiting spring 53 is elastically deformed to prevent further transmission of the handle operating force. In consequence, the force from the jaw unit 24 that acts on the operating portion 9c of the vibration transmitting member 9 can never be excessive, so that excessive displacement of the operating portion 9c can be prevented to maintain functions for incision and coagulation.

As shown in FIG. 1, moreover, an electrode mounting portion 56 for the connection of a high-frequency cable is formed on the vibrator connecting portion 11 of the handling portion body 12. As shown in FIG. 2, an electrode pin mounting hole 57 is formed in the electrode mounting portion 56. The electrode pin 15 is attached to the electrode pin mounting hole 57. A fixing screw 15b is formed on the proximal end portion of a pin body 15a of the electrode pin 15. Further, a connecting portion 15c for the connection of a high-frequency cable (not shown) is formed on the distal end portion of the pin body 15a. With an electrode insulating cover 58 mounted on an intermediate portion of the pin body 15a, the electrode pin 15 is attached to the electrode pin mounting hole 57 by means of the fixing screw 15b. A conic point portion 15d is formed on the opposite side of the electrode pin 15 to the connecting portion 15c.

Further, the inner peripheral surface of the proximal end portion of the handling portion body 12 is formed having a tapped hole portion 59 for mounting a retaining member to which the unit joint portion 6 of the vibrator unit 4 is releasably anchored as the vibrator unit 4 is coupled thereto. A substantially ring-shaped connecting member 60, formed of a conductive material such as metal, and a fixing ring 61 are successively screwed into the tapped hole portion 59.

Furthermore, the connecting member 60 is provided with an outer tube portion 60a, an inner tube portion 60b projecting backward beyond the outer tube portion 60a, and a joint portion 60c connecting the outer tube portion 60a and the inner tube portion 60b. The outer peripheral surface of the outer tube portion 60a of the connecting member 60 is formed having an external thread portion 60al that mates with the tapped hole portion 59 of the handling portion body 12. The connecting member 60 is attached to the tapped hole portion 59 of the handling portion body 12 by means of the external thread portion 60al so that its position is adjustable in the axial direction. After its position is adjusted, the connecting member 60 is fixed by means of the fixing ring 61 in the tapped hole portion 59 of the handling portion body 12. The electrode pin 15 is designed so that the point portion 15d can be butted for conduction against the external thread portion 60al on the outer periphery of the connecting member 60.

Further, a substantially conic engaging protuberance 61a protrudes from the inner peripheral surface of the proximal end portion of the fixing ring 61. In assembling the handle unit 2, probe unit 3, and vibrator unit 4 of the ultrasonic operating apparatus 1, the probe unit 3 and the vibrator unit 4 are integrally combined in advance, and the resulting combined unit is then combined with the handle unit 2. As this is done, the combined unit of the probe unit 3 and the vibrator unit 4 is inserted into the handle unit 2 through a rear end opening of the inner tube portion 60b of the connecting member 60, and is then inserted into the inner pipe 28 of the insert portion covering tube 19.

As shown in FIG. 1, the operating portion 9c on the extreme distal end portion of the probe unit 3 projects forward from the insert sheath portion 2a, and is set in a state such that it can seize the organism tissue between itself and the jaw unit 24. In this state, the unit joint portion 6 of a hand piece 5 of the vibrator unit 4 can be removably coupled to the vibrator connecting portion 11 of the handling portion body 12 of the handle unit 2.

In coupling the unit joint portion 6, moreover, the unit joint portion 6 is inserted along the outer peripheral surface of the inner tube portion 60b of the connecting member 60 toward the distal end, as shown in FIG. 2. At this time, the engaging ring 8 of the unit joint portion 6 is elastically deformed as it gets over the engaging protuberance 61a of the fixing ring 61 of the vibrator connecting portion 11. When the distal end face of the unit joint portion 6 engages a contact surface of the joint portion 60c on the proximal end side of the connecting member 60, the engaging ring 8 is pressed against the engaging protuberance 61a of the fixing ring 61 by elastic force, thereby generating frictional force. Thereupon, the unit joint portion 6 is detachably fixed to the vibrator connecting portion 11.

Located in the handling portion body 12 is a cylindrical conductive tube 62 of a conductive material such as metal that electrically conducts to the connecting member 60. The conductive tube 62 is formed having a plurality of slits that axially extend from an intermediate portion toward the proximal end portion and are arranged in the circumferential direction. A flange-shaped engaging protuberance 62a protrudes outward from the proximal end portion of the conductive tube 62. The engaging protuberance 62a is coupled it is inserted and fitted in an engaging groove portion 60d of the inner tube portion 60b of the connecting member 60 by means of the spring force of the conductive tube 62. Thus, the conductive tube 62 is supported on the connecting member 60 so as to be rotatable around the axis and fixed in the axial direction. Formed on the distal end side of the conductive tube 62, moreover, is a small-diameter tube portion 62b that is inserted in the slider mounting member 51. The inside diameter of the small-diameter tube portion 62b is greater than a maximum diameter on the proximal end side of the vibration transmitting member 9, that is, the diameter of a maximum-diameter portion 9e of the proximal end portion of a horn portion 9d. When the slider mounting member 51 moves in the axial direction as the slider 54 is slid to open or close the movable 15 handle 14, the slider mounting member 51 slides along the small-diameter tube portion 62b of the conductive tube 62.

Positioning flat portions 9f1 and 9f2, which are obtained by cutting opposite side faces of a circular cross section flat, as shown in FIG. 5D, are formed in a position for a node of vibration on the extreme proximal end side of the vibration transmitting member 9, as shown in FIG. 5A. Formed in this position is an odd-profile portion 9g having a noncircular cross section.

Further, a ring-shaped conductive member 63 of conductive material rubber, such as conductive silicone rubber, is attached to the inner peripheral surface of the distal end portion of the small-diameter tube portion 62b of the conductive tube 62 in a position near a node of vibration of the vibration transmitting member 9. An odd-shaped hole portion 63a corresponding to the odd-profile portion 9g of the vibration transmitting member 9 is formed in the inner peripheral surface of the conductive member 63. The odd-shaped hole portion 63a is formed having a circular hole portion 63b corresponding to a circular profile portion of the vibration transmitting member 9 and flat portions 63c1 and 63c2 corresponding to the flat portions 9f1 and 9f2, respectively. In assembling the ultrasonic operating apparatus 1, the odd-profile portion 9g of the vibration transmitting member 9 is caused to engage the odd-shaped hole portion 63a of the conductive member 63. This engaging portion forms a first dislocation preventing portion 64 for preventing dislocation between the respective joint surfaces of the vibration transmitting member 9 and the conductive member 63 in the rotating direction.

As shown in FIG. 3, moreover, positioning flat portions 62c1 and 62c2, which are obtained by cutting opposite side faces of a circular cross section flat, are formed on the outer peripheral surface of the small-diameter tube portion 62b of the conductive tube 62. Formed in this position is an odd-profile portion 62d having a noncircular cross section.

Further, an odd-shaped hole portion 51b corresponding to the odd-profile portion 62d of the conductive tube 62 is formed in the inner peripheral surface of the slider mounting member 51. The odd-shaped hole portion 51b is formed having a circular hole portion 51c corresponding to a circular profile portion of the small-diameter tube portion 62b of the conductive tube 62 and flat portions 51d1 and 51d2 corresponding to the flat portions 62c1 and 62c2, respectively. In assembling the ultrasonic operating apparatus 1, the odd-profile portion 62d of the conductive tube 62 is caused to engage the odd-shaped hole portion 51b of the slider mounting member 51. This engaging portion forms a second dislocation preventing portion 65 for preventing dislocation between the respective joint surfaces of the conductive tube 62 and the slider mounting member 51 in the rotating direction.

Thus, as the rotary knob 20 rotates, the force to rotate the rotary knob 20 is transmitted from the rotating barrel portion 42c that rotates integrally with the eccentric barrel 42 to the drive shaft connecting member 46 and the slider mounting member 51 via the rotary fixing pin 48, and then transmitted to the conductive tube 62 via the second dislocation preventing portion 65. Further, this handling force is transmitted to the vibration transmitting member 9 via the first dislocation preventing portion 64, whereupon the operating portion 9c and the jaw unit 24 are simultaneously rotated around the axis. While the rotary knob 20 is rotating, dislocation between the respective joint surfaces of the operating portion 9c and the jaw unit 24 in the rotating direction is prevented in a manner such that the jaw unit 24 is closed and joined to the operating portion 9c of the vibration transmitting member 9 by means of the second dislocation preventing portion 65 between the conductive tube 62 and the slider mounting member 51 and the first dislocation preventing portion 64 between the vibration transmitting member 9 and the conductive member 63.

Further, a second odd-profile portion 9h is formed in a position for a node of vibration near the extreme distal end portion of the vibration transmitting member 9. As shown in FIG. 5B, the second odd-profile portion 9h is formed having positioning flat portions 9i1 and 9i2 that are obtained by cutting opposite side faces of a circular cross section flat.

Furthermore, a spanner catch portion 9j for a driving tool is formed on the proximal end portion of the vibration transmitting member 9. As shown in FIG. 5E, the spanner catch portion 9j is formed having positioning flat portions 9k1 and 9k2 that are obtained by cutting opposite side faces of a circular cross section flat.

Further, an engaging hole portion 32c that engages the second odd-profile portion 9h of the vibration transmitting member 9 is formed on a tube wall portion corresponding to the second odd-profile portion 9h of the vibration transmitting member 9, that is, the inner peripheral surface of the coupling pipe 32, as shown in FIG. 8C. The engaging hole portion 32c is formed having positioning flat portions 32c1 and 32c2 that are obtained by flattening opposite side faces of a circular cross section to match the second odd-profile portion 9h of the vibration transmitting member 9. In assembling the ultrasonic operating apparatus 1, the second odd-profile portion 9h of the vibration transmitting member 9 is caused to engage the engaging hole portion 32c of the coupling pipe 32. This engaging portion forms a third dislocation preventing portion 67 for preventing dislocation between the respective joint surfaces of the vibration transmitting member 9 and the coupling pipe 32.

Furthermore, a retaining ring 68 of a plastic material is fitted in the inner peripheral surface of the drive shaft connecting member 46. The inner peripheral surface of the retaining ring 68 has a diameter smaller than the inside diameter of the drive shaft connecting member 46. Thus, the metallic drive shaft connecting member 46 can be prevented from directly touching the vibration transmitting member 9.

Further, a sealing rubber ring 69 is attached to the flange portion 51a of the slider mounting member 51. The rubber ring 69 serves to maintain airtightness between the slider mounting member 51 and the small-diameter tube portion 62b of the conductive tube 62.

The following is a description of the functions of the configuration described above. The ultrasonic operating apparatus 1 of the present embodiment can be disassembled into three units, the handle unit 2, probe unit 3, and vibrator unit 4. In working the ultrasonic operating apparatus 1, the mounting screw 9a of the probe unit 3 is previously driven into and fixed to the internal thread portion of the tapped hole portion 7a of the vibrator unit 4, whereby the probe unit 3 and the vibrator unit 4 in the disassembled state are joined together. Thereafter, the integrated unit of the probe unit 3 and the vibrator unit 4 is attached to the handle unit 2.

In the operation for the attachment to the handle unit 2, the probe unit 3 is inserted into the handling portion body 12 through the rear end opening of the inner tube portion 60b of the connecting member 60 at the vibrator connecting portion 11 of the handling portion body 12 of the handle unit 2. Then, it is inserted into the inner pipe 28 of the insert portion covering tube 19.

As shown in FIG. 1, the operating portion 9c on the extreme distal end portion of the probe unit 3 is projected ahead of the insert sheath portion 2a. Thereupon, it can be set in a state such that the organism tissue can be held between itself and the jaw unit 24. As this is done, the unit joint portion 6 of the hand piece 5 of the vibrator unit 4 is removably coupled to the vibrator connecting portion 11 of the handling portion body 12 of the handle unit 2.

In coupling the unit joint portion 6, moreover, the unit joint portion 6 is inserted along the inner tube portion 60b of the connecting member 60 toward the distal end, as shown in FIG. 2. At this time, the engaging ring 8 of the unit joint portion 6 is elastically deformed as it gets over the engaging protuberance 61a of the fixing ring 61 of the vibrator connecting portion 11. When the distal end face of the unit joint portion 6 engages the contact surface of the joint portion 60c on the proximal end side of the connecting member 60, the engaging ring 8 of the hand piece 5 is pressed against the engaging protuberance 61a of the fixing ring 61 by an elastic force, thereby generating frictional force. Thereupon, the portions are detachably fixed. Forces in two directions, radial and axial, are generated in the respective contact portions of the engaging ring 8 and the engaging protuberance 61a of the fixing ring 61. The contact portions are firmly fixed in both axial and circumferential directions by means of a frictional force and engaging force that are produced by the forces in the two directions. In this state, the operation for assembling the handle unit 2, probe unit 3, and vibrator unit 4 in the combined state shown in FIG. 1 is finished.

In assembling the ultrasonic operating apparatus 1, the vibration transmitting member 9 is positioned in the inner pipe 28 by means of a plurality of rubber rings 9b that are set individually in positions for nodes of ultrasonic vibration of the vibration transmitting member 9. As this is done, the metallic inner pipe 28 is prevented from directly touching the vibration transmitting member 9 by means of the rubber rings 9b.

In assembling the ultrasonic operating apparatus 1, moreover, the odd-profile portion 62d of the conductive tube 62 is caused to engage the odd-shaped hole portion 51b of the slider mounting member 51. This engaging portion forms the second dislocation preventing portion 65 for preventing dislocation between the respective joint surfaces of the conductive tube 62 and the slider mounting member 51 in the rotating direction. Likewise, the odd-profile portion 9g of the vibration transmitting member 9 is caused to engage the odd-shaped hole portion 63a of the conductive member 63. This engaging portion forms the first dislocation preventing portion 64 for preventing dislocation between the respective joint surfaces of the vibration transmitting member 9 and the conductive member 63 in the rotating direction. Further, the second odd-profile portion 9h of the vibration transmitting member 9 is caused to engage the engaging hole portion 32c of the coupling pipe 32. This engaging portion forms the third dislocation preventing portion 67 for preventing dislocation between the respective joint surfaces of the vibration transmitting member 9 and the coupling pipe 32.

In working the ultrasonic operating apparatus 1, furthermore, the movable handle 14 is operated with the stationary handle 13 of the handle unit 2 gripped. As the movable handle 14 is operated in this manner, the handling rod 30 moves in the insert sheath portion 2b, thereby opening or closing the jaw body 24a that is attached to the tip 25 of the distal working portion 2a.

If the operation (closing operation) for gripping the movable handle 14 is carried out, the actuator pins 18 are rotated in the clockwise direction of FIG. 1 around the handle pivots 17. As this is done, the actuator pins 18 are advanced substantially straight to the distal end side within the ranges of their movement. This motion of the actuator pins 18 is transmitted to the slider 54 via the engaging portions between the actuator pins 18 and front and rear wall surfaces of the engaging groove 54a of the slider 54, whereupon the slider 54 is moved to the distal end side.

Further, this advancing motion of the slider 54 is transmitted from the slider mounting member 51 to the drive shaft connecting member 46 by means of the fixing screws 52. The handling rod 30 is pushed out toward the distal end by means of the drive shaft connecting member 46. Thereupon, the handling rod 30 advances in the insert portion covering tube 19. In consequence, the jaw unit 24 is fully closed with the tip 25 of the jaw unit 24 pressed against the operating portion 9c of the vibration transmitting member 9, as indicated by the solid line in FIG. 7. When the jaw unit 24 is in its fully-closed position, the seizing surface 25d on the underside of the tip 25 is intimately in contact with the contact surface 9m of the operating portion 9c of the vibration transmitting member 9 without a gap. In this state, the object of operation is held and pressurized between the tip 25 of the jaw unit 24 on the distal end of the handle unit 2 and the operating portion 9c, for use as an ultrasonic probe, on the distal end of the vibration transmitting member 9 of the probe unit 3. The object of operation is coagulated and incised with frictional heat that is generated by ultrasonic vibration.

When the object of operation is subjected to ultrasonic operation, moreover, the organism tissue is securely held to generate frictional heat with the jaw unit 24 closed, so that the operating portion 9c of the vibration transmitting member 9 is bent downward by a force of pressure from the tip 25. As this is done, the tip 25 swings around the supporting pins 27 of the jaw body 24a. Thus, the tip 25 can be pressed vertically against the inclined operating portion 9c. In consequence, the organism tissue can be securely coagulated and incised throughout the length of the tip 25.

Further, the limiting spring 53 is compressed to be shorter than its free length and subjected to the equipment load as it is mounted. When the movable handle 14 is closed, the jaw unit 24 can be directly opened or closed without subjecting the limiting spring 53 to elastic deformation with a handle operating force lighter than the equipment load. Thus the handling can be improved.

If a force heavier than the equipment load of the limiting spring 53 is applied when the movable handle 14 is closed, the limiting spring 53 is elastically deformed to prevent further transmission of the handle operating force. In consequence, the force from the jaw unit 24 that acts on the operating portion 9c of the vibration transmitting member 9 can never be excessive, so that excessive displacement of the operating portion 9c can be prevented to maintain the functions for incision and coagulation.

When the movable handle 14 in the fully-closed position is opened, moreover, the actuator pins 18 are rotated in the counterclockwise direction of FIG. 1 around the handle pivots 17. As the actuator pins 18 are moved in this manner, the slider 54 is moved backward.

This retreating motion of the slider 54 is transmitted from the slider mounting member 51 to the drive shaft connecting member 46 by means of the fixing screws 52. The handling rod 30 is pulled backward by means of the drive shaft connecting member 46. Thereupon, the handling rod 30 retreats in the insert portion covering tube 19, and a coupling pin 36 of a connecting member 34, along with the handling rod. 30, also retreats parallel to the central axis of the insert portion covering tube 19. As this is done, the coupling pin 36 retreats sliding in the coupling pin 24d of the jaw body 24a. Thereupon, the tip 25 of the jaw unit 24 moves away from the vibration transmitting member 9, that is, the jaw unit 24 turns clockwise around the pivot pins 33, thereby opening with respect to the operating portion 9c of the vibration transmitting member 9, as indicated by imaginary line in FIG. 7.

When the rotary knob 20 is rotated, moreover, the force to rotate the rotary knob 20 is transmitted from the rotating barrel portion 42c rotating integrally with the rotating barrel portion 42c to the drive shaft connecting member 46 via the rotary fixing pin 48. Thus, the members that include the insert portion covering tube 19 and the members therein, the eccentric barrel 42 and the rotating barrel portion 42c mounted on the proximal end portion of the insert portion covering tube 19, and the rotary knob 20 can rotate integrally with the drive shaft connecting member 46 with respect to the handling portion body 12. Further, the force to rotate the rotary knob 45 is transmitted from the rotating barrel portion 42c to the drive shaft connecting member 46 via the rotary fixing pin 48, whereupon the slider mounting member 51, limiting spring 53, and slider 54 also rotate integrally with one another. Thus, the handling rod 30 can be prevented from being twisted.

As the rotary knob 20 rotates, furthermore, the force to rotate the rotary knob 20 is transmitted from the rotating barrel portion 42c that rotates integrally with the eccentric barrel 42 to the drive shaft connecting member 46 and the slider mounting member 51 via the rotary fixing pin 48, and then transmitted to the conductive tube 62 via the second dislocation preventing portion 65. Further, this handling force is transmitted to the vibration transmitting member 9 via the first dislocation preventing portion 64, whereupon the operating portion 9c and the jaw unit 24 are simultaneously rotated around the axis. While the rotary knob 20 is rotating, dislocation between the conductive tube 62 and the slider mounting member 51 in the rotating direction is prevented by means of the second dislocation preventing portion 65. Further, dislocation between the vibration transmitting member 9 and the conductive member 63 in the rotating direction is prevented by means of the first dislocation preventing portion 64. Furthermore, dislocation between the vibration transmitting member 9 and the coupling pipe 32 in the rotating direction is prevented by means of the third dislocation preventing portion 67. Thus, dislocation between the respective joint surfaces of the operating portion 9c and the jaw unit 24 in the rotating direction is prevented in a manner such that the jaw unit 24 is closed and joined to the operating portion 9c of the vibration transmitting member 9.

High-frequency current supplied from a high-frequency cable that is connected to the connecting portion 15c of the electrode pin 15 flows from the point portion 15d to the connecting member 60. Further, it flows through the conductive member 63 of conductive rubber and reaches the vibration transmitting member 9. Thereafter, it is discharged from the distal end of the operating portion 9c to carry out high-frequency operation.

The jaw holding member 31 and the outer pipe 21 of the insert portion covering tube 19 are metallic and electrically conductive. Further, the jaw holding member 31 and the insert portion covering tube 19 are pre-insulated by means of the coupling pipe 32 and the insulating tube 22, respectively. Thus, the high-frequency current is prevented from flowing to parts other than the object of operation.

For reuse sake, moreover, the ultrasonic operating apparatus 1 of the present embodiment is disassembled into three units, the handle unit 2, probe unit 3, and vibrator unit 4, after use. By doing this, each of the disassembled units including the handle unit 2, probe unit 3, and vibrator unit 4 can be positively cleaned with a brush or the like. Thus, the convenience of cleaning of the ultrasonic operating apparatus 1 can be improved.

In the jaw unit 24 of the present embodiment, moreover, the supporting pins 27 of the jaw body 24a can be drawn out of the insertion holes 101 and 102 of the tip 25 and the seizing portion mounting member 26, individually, in a manner such that the respective distal end portions of the arms 24b1 and 24b2 of the jaw body 24a are bent outward. Thus, the tip 25 and the seizing portion mounting member 26 of the jaw unit 24 can be removed from the jaw body 24a. If the tip 25 is worn away during use, therefore, the worn tip 25 is removed from between the respective distal end portions of the arms 24b1 and 24b2 of the jaw body 24a, and a new tip 25 is mounted between the respective distal end portions of the arms 24b1 and 24b2. By doing this, the tip 25 can be replaced with ease.

The above-described configuration produces the following effects. More specifically, in the present embodiment, the tip 25 and seizing portion mounting member 26 of the jaw unit 24 are removably coupled between the respective distal end portions of the arms 24b1 and 24b2 of the jaw body 24a. If the tip 25 is worn away, therefore, a new tip 25 can be mounted between the respective distal end portions of the arms 24b1 and 24b2 of the jaw body 24a after the worn tip 25 is removed from between the respective distal end portions of the arms 24b1 and 24b2 of the jaw body 24a. In consequence, the tip 25 can be replaced with ease. If the tip 25 of the jaw unit 24 is worn away and rendered unusable, the cost of parts replacement can be made lower than in the conventional case where all the parts that are assembled to the jaw unit 24 and unitized are replaced, and the running cost of the whole system of the ultrasonic operating apparatus 1 can be lowered.

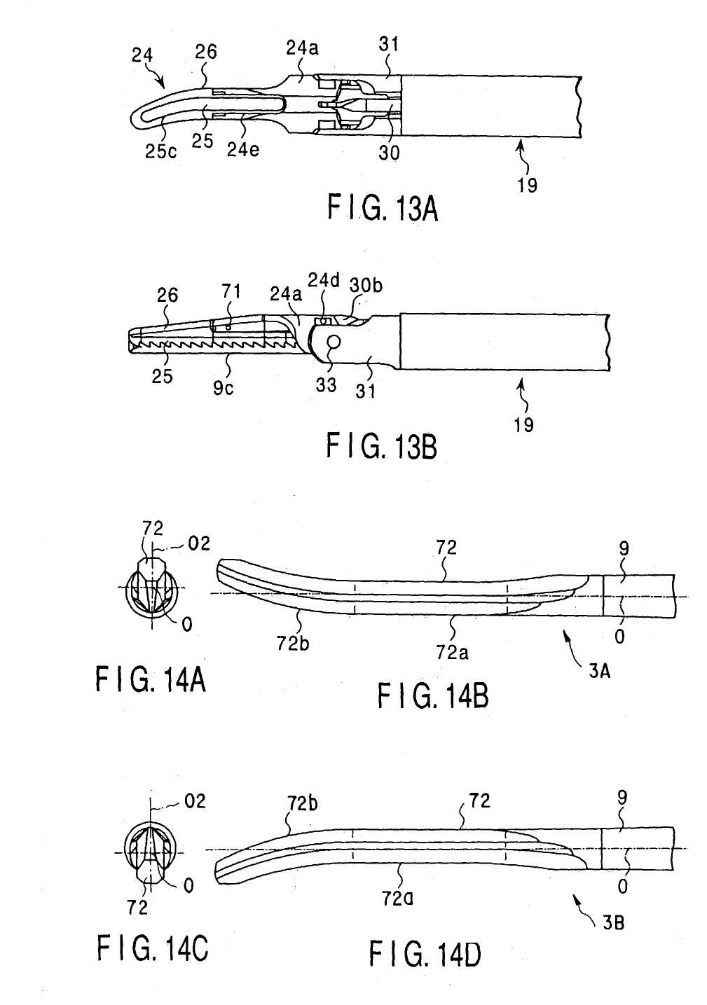

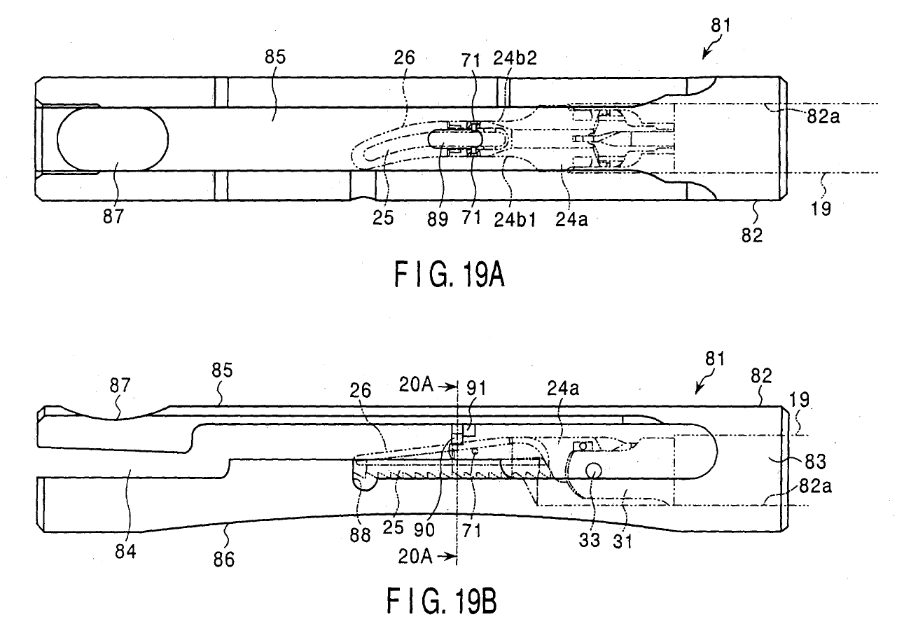

FIGS. 13A to 20B show a second embodiment of the present invention. The present embodiment is obtained by modifying the configuration of the ultrasonic operating apparatus 1 of the first embodiment (see FIGS. 1 to 12) in the following manner.

More specifically, the ultrasonic operating apparatus 1 of the present embodiment comprises a vibration transmitting member 9 having a distal end operating portion 72, as shown in FIGS. 14A and 14B. The operating portion 72 is provided with a straight portion 72a, which is extends substantially in a straight line along a center line 0 of a probe unit 3, and a curved portion 72b, which is gently curved in a circular arc to be deviated from the center line 0 of the probe unit 3. The curved portion 72b is formed on the distal end portion of the straight portion 72a.

As shown in FIG. 14A, moreover, the curved portion 72b is formed axisymmetrically with respect to the direction of a straight line 02 in which a jaw unit 24 is opened or closed. By inserting the probe unit 3 into a handle unit 2, as shown in FIGS. 14A and 14B, therefore, a rightward first probe unit 3A can be formed having the curved portion 72b curved in a rightward circular arc. By inserting the probe unit 3 into the handle unit 2 in a 180°-turned manner, on the other hand, a leftward second probe unit 3B can be formed having the distal end operating portion 72 of the vibration transmitting member 9 curved in a leftward circular arc, as shown in FIGS. 14C and 14D.

As shown in FIG. 17, moreover, a jaw body 24a of the jaw unit 24 is provided with a pair of arms 24b1 and 24b2, which are symmetrical with respect to the central axis of an insert portion and have pin insertion holes 74 in their respective distal end portions, individually. Supporting pins (support shaft portions) 71 for supporting a tip 25 are inserted in the pin insertion holes 74, individually. The respective distal end portions of the supporting pins 71 protrude inward from the arms 24b1 and 24b2, individually. Further, the respective proximal end portions of the supporting pins 71 are fixed in the respective pin insertion holes 74 of the arms 24b1 and 24b2, individually. On the distal end side of the pin insertion holes 74, furthermore, straight grooves 75 individually extend along the center line 0 of the probe unit 3B inside the arms 24b1 and 24b2.

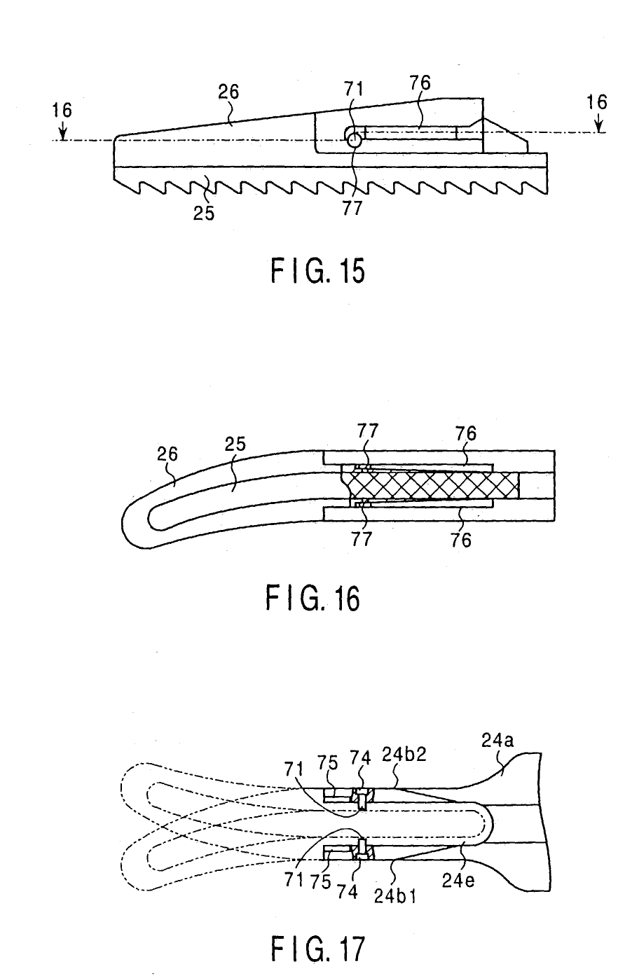

As shown in FIG. 15, that part of the tip 25 of the jaw unit 24 which is inserted in the slit 24e between the arms 24b1 and 24b2 of the jaw body 24a is provided with guide grooves 76 and mounting holes 77 for the supporting pins 71. The mounting holes 77 are located substantially in the central region of the tip 25 with respect to its longitudinal direction.

As shown in FIG. 16, moreover, the guide grooves 76 extend from the rear end position of the tip 25 to the position for the mounting holes 77. In attaching the tip 25 to the jaw body 24a, the supporting pins 71 are guided along the guide grooves 76 to the mounting holes 77, individually.

Further, each guide groove 76 is formed having a taper surface such that the groove depth gradually decreases from the rear end position of the tip 25 toward each mounting hole 77. The mounting hole 77 is located in a position where the groove depth of the guide groove 76 is minimal. Formed at the junction of the guide groove 76 and the mounting hole 77 is a click step for preventing the supporting pin 71 from slipping out of the mounting hole 77. Thus, in attaching the tip 25 to the jaw body 24a, the supporting pins 71 on the opposite sides are moved away from each other as the supporting pins 71 are moved along the respective taper surfaces of the guide grooves 76 toward the mounting holes 77. Thereupon, the supporting pins 71 get over the click steps and are removably inserted into the mounting holes 77.

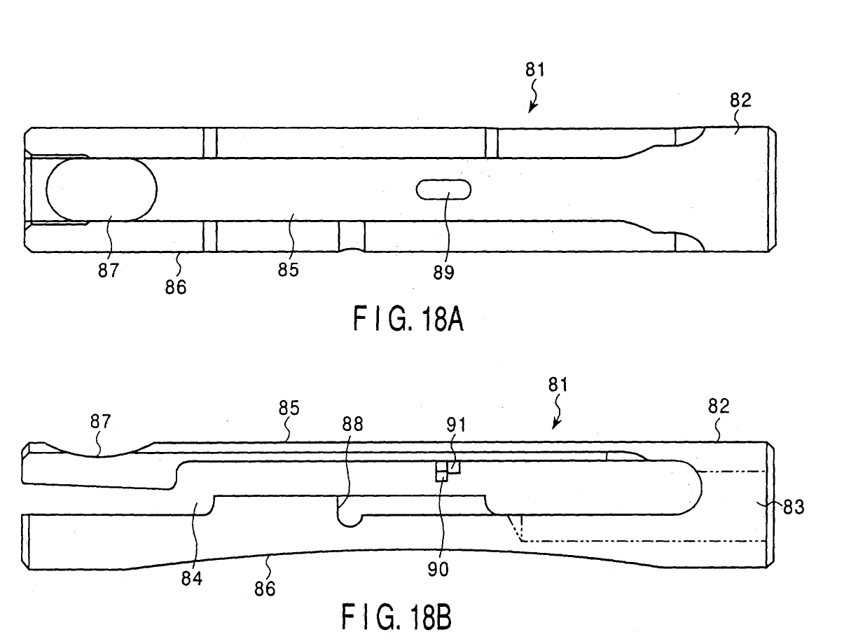

Provided according to the present embodiment, moreover, is a tip changing tool 81 shown in FIGS. 18A an 18B, which is used to remove the tip 25 from the jaw body 24a. A jig body 82 of the changing tool 81 is provided with an insertion hole 83 into which a distal working portion 2b of the ultrasonic operating apparatus 1 can be inserted and a stopper portion 88 for locating the position of insertion of the distal working portion 2b inserted in the insertion hole 83.

Furthermore, one end portion of a handling arm 85 is coupled to the inlet side of the insertion hole 83 of the jig body 82. As shown in FIG. 18B, a gap 84 of a given width is formed between the handling arm 85 and the jig body 82, covering the other region than their junction. The handling arm 85 is supported on the jig body 82 so as to be rockable around the junction as a hinge portion.

Further, a handgrip depression 86 is formed in the peripheral wall surface of the jig body 82 on the side opposite from the handling arm 85. Furthermore, a finger-rest depression 87 is formed on the free end side of the handling arm 85.

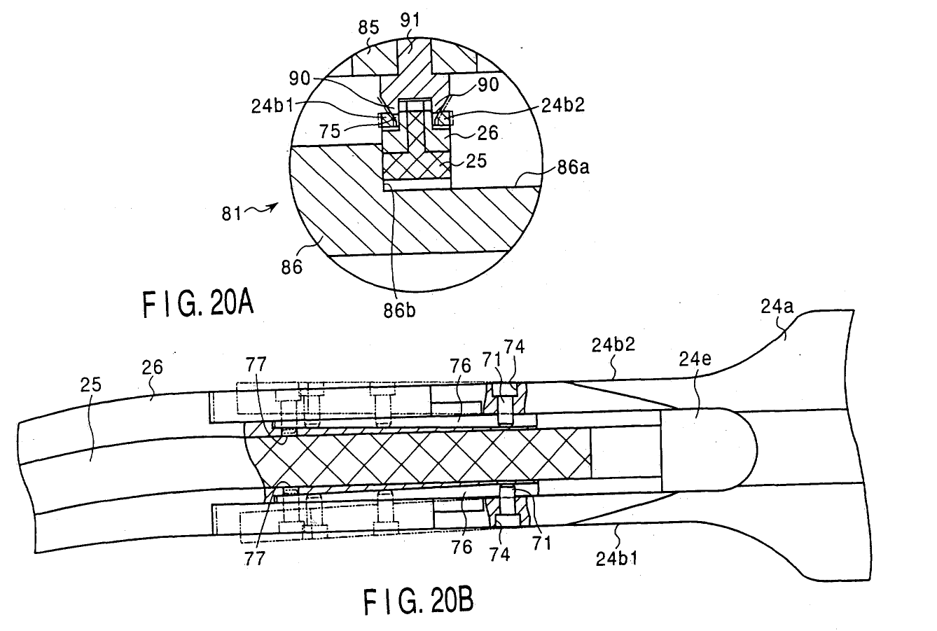

Further, a separating portion 89 is provided in the middle portion of the handling arm 85. As shown in FIG. 20A, the separating portion 89 is provided with a projecting member 91 that protrudes from the inner peripheral surface of the handling arm 85 toward the jig body 82. The distal end portion of the projecting member 91 is provided with a pair of wedge-shaped separating claws 90, left and right, which are spaced and opposed to each other. The separating claws 90 can be removably inserted into spaces between the tip 25 and the arms 24b1 and 24b2 on the opposite sides of the jaw body 24a of the distal working portion 2b as the handling arm 85 rocks. As the separating claws 90 are inserted into the spaces between the tip 25 and the arms 24b1 and 24b2, the arms 24b1 and 24b2 are individually pushed out and elastically deformed in a direction such that the space between the arms 24b1 and 24b2 widens. As the arms 24b1 and 24b2 are elastically deformed, the respective supporting pins 71 of the arms 24b1 and 24b2 are pushed out individually from the mounting holes 77 of the tip 25, as indicated by imaginary lines in FIG. 20B. As this is done, the respective supporting pins 71 of the arms 24b1 and 24b2 pass over the click steps and are drawn out of the mounting holes 77. Thereupon, the respective supporting pins 71 of the arms 24b1 and 24b2 are disengaged from the mounting holes 77 of the tip 25.

The following is a description of the functions of the present embodiment arranged in this manner. In attaching the tip 25 to the jaw body 24a, according to the present embodiment, the respective supporting pins 71 of the arms 24b1 and 24b2 are inserted into the guide grooves 76 of the tip 25 through rear end openings of the guide grooves 76, as shown in FIG. 15. As this is done, the respective supporting pins 71 of the arms 24b1 and 24b2 are guided along the guide grooves 76 to the mounting holes 77, individually.

As the respective supporting pins 71 of the arms 24b1 and 24b2 move, the supporting pins 71 are moved along the respective taper surfaces of the guide grooves 76 toward the distal ends. As this is done, the supporting pins 71 are moved along the respective taper surfaces of the guide grooves 76 in a direction such that the space between the supporting pins 71 on the opposite sides widens. Then, the supporting pins 71 get over the click steps and are removably inserted into the mounting holes 77 for engagement, whereupon the tip 25 is attached to the jaw body 24a.

Further, the tip changing tool 81 is used to remove the tip 25 from the jaw body 24a of the jaw unit 24. In working the tip changing tool 81, the position of insertion of the distal working portion 2b of the ultrasonic operating apparatus 1 is located by means of the stopper portion 88 with the distal working portion 2b inserted in the insertion hole 83 of the tip changing tool body 82. In this state, the handling arm 85 is rocked around the hinge portion on the inlet side of the insertion hole 83 of the jig body 82 with respect to the jig body 82. As the handling arm 85 is rocked in this manner, it is inserted into the space between the tip 25 and the arms 24b1 and 24b2 on the opposite sides of the jaw body 24a of the distal working portion 2b. As the separating claws 90 are inserted into the spaces between the tip 25 and the arms 24b1 and 24b2, the arms 24b1 and 24b2 are individually pushed out and elastically deformed in a direction such that the space between the arms 24b1 and 24b2 widens. As the arms 24b1 and 24b2 are elastically deformed, the respective supporting pins 71 of the arms 24b1 and 24b2 are pushed out individually from the mounting holes 77 of the tip 25, as indicated by the imaginary lines in FIG. 20B. As this is done, the respective supporting pins 71 of the arms 24b1 and 24b2 get over the click steps and are drawn out of the mounting holes 77. Thereupon, the respective supporting pins 71 of the arms 24b1 and 24b2 are disengaged from the mounting holes 77 of the tip 25. If the jig 81 is pulled toward the distal end in this state, the tip 25 can be removed integrally with the tip changing tool body 82 from the jaw body 24a of the jaw unit 24.

The above-described configuration produces the following effects. More specifically, in the present embodiment, the tip 25 of the jaw unit 24 is removably coupled between the respective distal end portions of the arms 24b1 and 24b2 of the jaw body 24a. If the tip 25 is worn away, therefore, a new tip 25 can be mounted between the respective distal end portions of the arms 24b1 and 24b2 of the jaw body 24a after the worn tip 25 is removed from between the respective distal end portions of the arms 24b1 and 24b2 of the jaw body 24a. In consequence, the tip 25 can be replaced with ease. If the tip 25 of the jaw unit 24 is worn away and rendered unusable, the cost of parts replacement can be made lower than in the conventional case where all the parts that are assembled to the jaw unit 24 and unitized are replaced, and the running cost of the whole system of the ultrasonic operating apparatus 1 can be lowered. Thus, since the tip 25 of the jaw unit 24 is of the replaceable type, more operations can be performed by only replacing low-priced parts, so that the cost can be lowered.

According to the present embodiment, moreover, the distal end operating portion 72 of the vibration transmitting member 9 is provided with the straight portion 72a and the curved portion 72b that is gently curved in a circular arc to be deviated from the center line 0 of the probe unit 3. As shown in FIG. 14A, the curved portion 72b is formed axisymmetrically with respect to the direction of the straight line 02 in which the jaw unit 24 is opened or closed. By inserting the probe unit 3 into the handle unit 2, as shown in FIGS. 14A and 14B, therefore, the rightward first probe unit 3A can be formed having the curved portion 72b curved in a rightward circular arc. By inserting the probe unit 3 into the handle unit 2 in a 180°-turned manner, on the other hand, the leftward second probe unit 3B can be formed having the distal end operating portion 72 of the vibration transmitting member 9 curved in a leftward circular arc, as shown in FIGS. 14C and 14D. A reversed operating device can be easily formed by attaching the jaw unit 24 that is curved in the same direction to match the shape of the probe unit 3. Thus, one probe unit 3 can be easily turned in two different directions, so that the number of types of operating devices to be assorted can be reduced and the cost can be lowered.

In removing the tip 25 from the jaw body 24a of the jaw unit 24 according to the present embodiment, furthermore, the tip 25 is removed integrally with the tip changing tool body 82 from the jaw body 24a of the jaw unit 24 by using the tip changing tool 81. Therefore, the operation for removing the particularly small-sized tip 25 from the jaw unit 24 can be carried out with ease, and this operation can be facilitated.

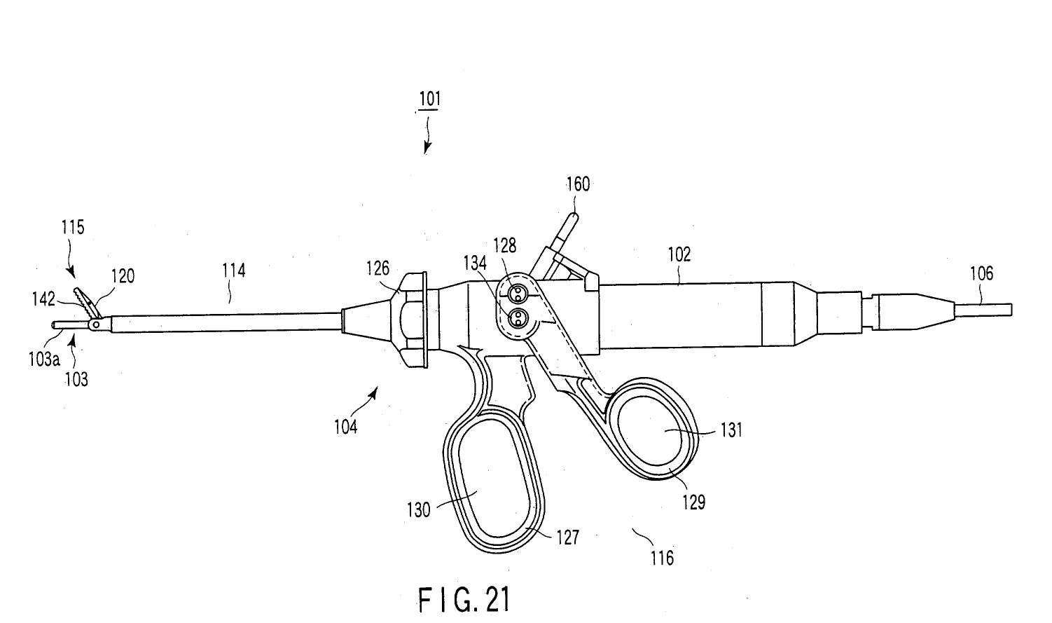

FIG. 21 to FIG. 30B show a third embodiment of the present invention. As shown in FIG. 21, a ultrasonic operating apparatus 101 according to the present embodiment is composed of: a vibrator unit 102; a probe (or probe unit) 103; and a handle unit 104. These units do not require a special work, and disassembling and predetermined assembling can be easily carried out.

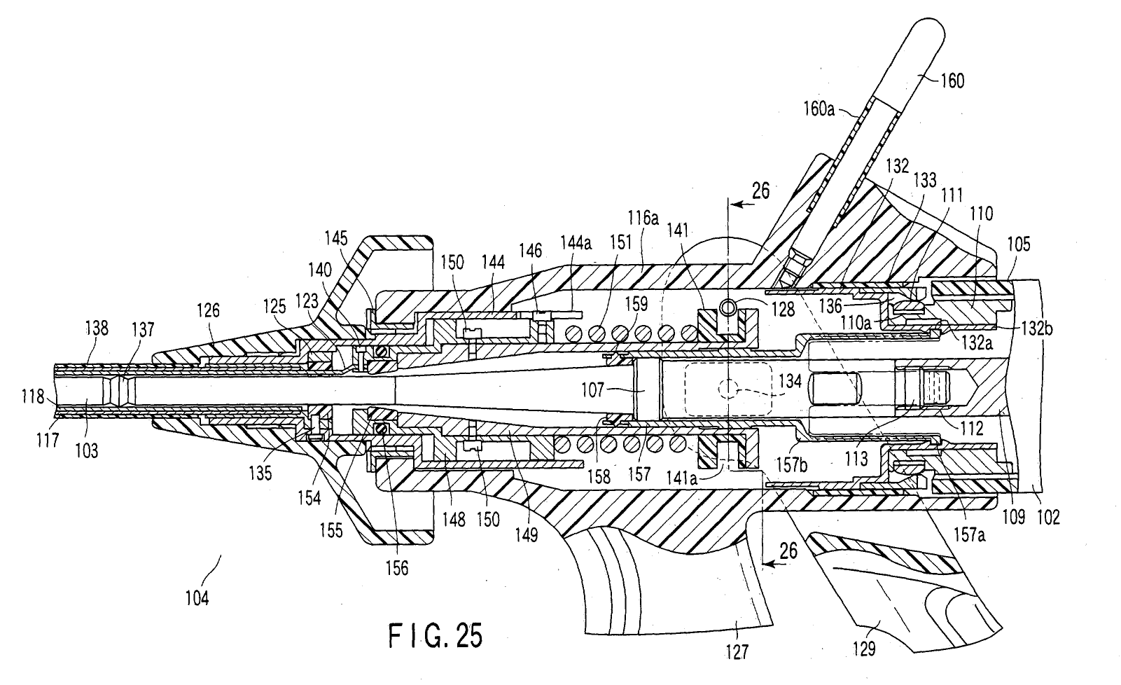

FIG. 25 shows an internal construction of the above vibrator unit 102. The vibrator unit 102 has a piezoelectric element (not shown) which converts a current into ultrasonic vibration. This piezoelectric element is covered with a vibrator cover 105. One end of a cable 106 is connected to a rear end of the vibrator cover 105. The other end of this cable 106 is connected to a power unit main body (not shown). Then, a drive current is supplied from the power unit main body to the piezoelectric element through the above cable 106.

In addition, a proximal end of a horn 109 shown in FIG. 25 is coupled with the vibrator unit 102 at a frontal end of the piezoelectric element. A proximal end of a probe 103 is coupled at a distal end portion of this horn 109. In addition, ultrasonic vibration generated at the above piezoelectric element is amplified via the horn 109 so as to be transmitted to the probe 103.

A ring shaped attachment 110 for connecting the above handle unit 104 is fixed to a frontal end of the above vibrator cover 105. A peripheral groove 110a is formed at the outer periphery of the frontal end of the attachment 110. A partially cutout metallic engaging ring 111 is mounted to be engaged with this peripheral groove 110a.

A scow hole 112 is provided at a distal end portion of the horn 109. A male screw portion 113 for threadly engaging the screw hole 112 of the above horn 109 is provided at a proximal end of the probe 103. Then, the male screw portion 113 of the probe 103 is threadly attached to the screw hole 112 of the horn 103, whereby the probe 103 and horn 109 are removably connected to each other on the same straight line. In this connection state, the entire length from the piezoelectric element to a distal end of the prove 103 is designed so as to be an integral multiple of a half wavelength of ultrasonic vibration.

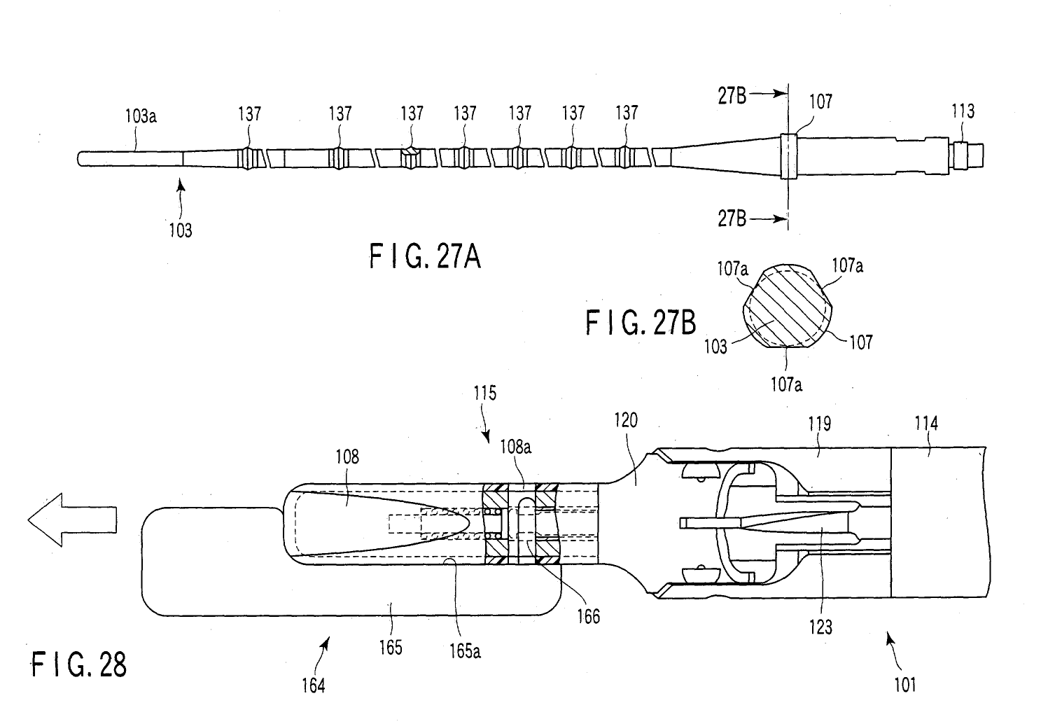

The probe 103 according to the present embodiment is formed in a straight shape from a proximal end to a distal end, as shown in FIG. 27A. A portion for reducing a sectional area in a direction orthogonal to an axial direction is formed at some portions of a node portion of vibration in the middle of the probe 103. In this manner, an amplitude required for operation is obtained at the distal end of the probe. In addition, a rubber ring 137 is mounted at some portions of the node portion of this vibration. Interference between the probe 103 and the handle unit 104 is prevented by this rubber ring 137.

In addition, a flange portion 107 is formed integrally with the node portion of vibration disposed at the most proximal end side of the probe 103. As shown in FIG. 27B, some portions of an outer periphery portion of this flange portion 107, three portions in the present embodiment, are cut out in a planar shape, and a flat portion 107a is provided.

As shown in FIG. 21, the above handle unit 104 has a longitudinally axis insert portion 114 and an operating portion 116. The insert portion 114 is inserted into a patient's cavity during surgical operation. The operating portion 116 is coupled with a proximal end of the insert portion 114. The operating portion 116 comprises a stationary handle 127 and a movable handle 129. The movable handle 129 is turnably mounted via a supporting pin 128 provided at the stationary handle 127. As shown in FIG. 26, a bush 153 consisting of a PTFE or the like with a low frictional coefficient is arranged at the outer periphery of the supporting pin 128. By this bush 153, sliding property of the movable handle 129 is improved.

As shown in FIG. 21, a finger hook portions 130 and 131 are provided at the stationary handle 127 and the movable handle 129, respectively. A surgeon seizes fingers of one hand with these finger hook portions 130 and 131, whereby the above movable handle 129 can be turned around the supporting pin 128. With a turning operation of this movable handle 129, a distal end acting portion 115 provided at a distal end of the above insert portion 114 is operated to be opened or closed.

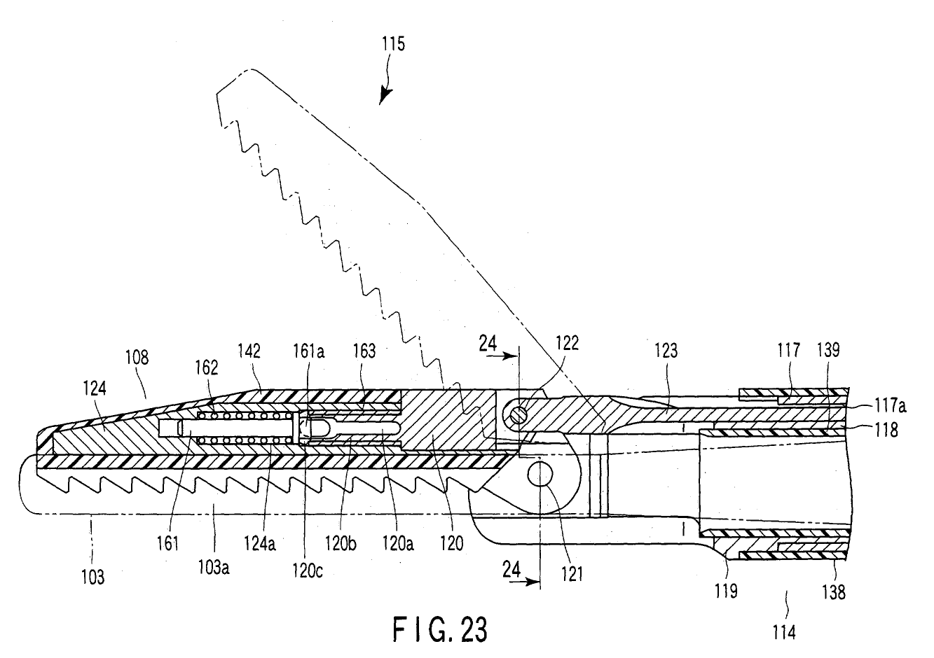

As shown in FIG. 23, the insert portion 114 has an outer pipe 117 extending from the operating portion 116. As shown in FIG. 25, a proximal end portion of the outer pipe 117 is fixed to a pipe fixing member 125. An inner pipe 118 is arranged inside of the outer pipe 117. An internal space of the inner pipe 118 forms a channel for routing the above probe 103.

As shown in FIG. 23, a channel 117a is formed between the outer pipe 117 and inner pipe 118 of the insert portion 114. A drive shaft (body) 123 for operating a distal end acting portion 115 is retractably inserted through this channel 117a. An electrical insulating tube 138 is covered on the outer periphery face of the above outer pipe 117.

As shown in FIG. 25, the pipe fixing member 125 of the insert portion 114 is fixed to a connecting member 144 via a fixing pin 135. The connecting member 144 is rotatably mounted axially around the insert portion relevant to a member of the fixing handle 127, by means of a fixing ring 145. A rotary knob 126 is mounted in a fixed state at the outer periphery portion of the distal end side of the connecting member 144. By rotating this rotary knob 126, the entire insert portion 114 including the above distal end acting portion 115 can be integrally rotated.

In addition, as shown in FIG. 25, a cylindrical drive force transmission intermediate member 148 and a cylindrical slider receptacle member 149 are disposed in the operating portion 116. A proximal end of a drive shaft 123 is connected to the drive force transmission intermediate member 148 via a drive force transmitting pin 140. The drive force transmission intermediate member 148 is mounted on the slider receptacle member 149 by means of a pin 150. A ring shaped slider member 141 and a coil shaped spring 151 are arranged at the outer periphery of the above slider receptacle member 149. The ring shaped slider member 141 is slidably mounted in an axial direction on the outer periphery of the slider receptacle member 149. The spring 151 is wound at the outer periphery of the slider receptacle member 149. This spring 151 is interposed between the slider member 141 and the drive force transmission intermediate member 148 to forwardly bias the drive force transmission intermediate member 148 with a predetermined quantity of equipment load.

A pin 146 is protruded on the outer periphery face of the proximal end portion of the above drive force transmission intermediate member 148. A slit 144a is formed at the proximal end portion of the connecting member 144 along an axial direction. The pin 146 is engaged to be fitted to this slit 144a. In this manner, in the connecting member 144 and the drive force transmission intermediate member 148, a relative axial periphery rotation is restricted by that pin 146, so that both of them integrally rotate in an axial periphery direction. However, only the drive force transmission intermediate member 148 can be moved in an axial direction.

As shown in FIG. 25, a packing 156 for sealing a gap is mounted between the connecting member 144 and an engaging portion of the drive force transmission intermediate member 148. Then, the leakage of abdominal gas or the like from the distal end side of the insert portion through the gap during surgical operation under laparoscopy is prevented by means of the packing 156. Interference proof rings 154 and 156 consisting of PTFE with low frictional coefficient are arranged inside of the rear end portion of the pipe fixing member 125 and the inside of the drive force transmission intermediate member 148. By these rings 154 and 155, the members 125 and 148 and the probe 103 are maintained so as not to be in contact with each other.

As shown in FIG. 26, flat portions 147 are formed at two portions facing each other on the inner periphery face of the proximal end portion of the slider receptacle member 149. In this manner, an abnormally shaped hole portion different from a circular shape is provided on the inner periphery shape of the proximal end portion of the slider receptacle member 149. A cylindrical contact member 157 consisting of an electrically conductive member is arranged to be engaged with the inside of the abnormally shaped hole portion of the slider receptacle member 149. The outer periphery face of the contact member 157 is formed in an external shape corresponding to a flat portion 147 of the abnormally shaped hole portion of the slider receptacle member 149.

As shown in FIG. 25, a ring shaped connecting rubber 158 consisting of electrically conductive rubbers is mounted at the distal end side portion of this contact member 157. This connecting rubber 158 is disposed near a node portion of vibration of the above prove 103. In addition, the probe 103 and contact member 157 are designed to be electrically connected by means of this electrically conductive connecting rubber 158. A protuberance 159 serving as packing is provided on the outer periphery face of this connecting rubber 158. This protuberance 159 prevents the leakage of abdominal gas or the like from the distal end of the insert portion through a gap during surgical operation under laparoscopy.

Further, a cylindrical portion 157b with a large diameter is provided at the rear end side of the contact member 157. This cylindrical portion 157b is formed so that it can be formed in a radial direction by providing one or a plurality of slits. A protuberance 157a is provided at the outer periphery of the rear end of this cylindrical portion 157b. This protuberance 157a is engaged with a peripheral groove 132a formed at a connecting member 132 described later. In addition, the outer periphery of a flange portion 107 of a probe 103 is designed so as to be engaged with the inner periphery face portion at the frontal end side of the above contact member 157. Thus, the contact member 157 is such that the inner periphery face portion at its frontal end side is formed in the same shape as the outer periphery of the flange portion 107.

Further, as shown in FIG. 25, a cylindrical handling portion housing 116a is provided at the upper end part of the stationary handle 127. Two cylindrical connecting members 132 and 133 disposed forwardly and backwardly are fixed on the inner periphery face of the rear end portion of this handling portion housing 116a. An engaging groove 136 for engagement with a vibrator unit 102 is formed between two connecting members 132 and 133. During assembling between the above handle unit 104 and vibrator unit 102, an attachment 110 of the vibrator unit 102 is inserted into a rear end opening of a handling portion housing 116a. At this time, an engaging ring 111 of the above vibrator unit 102 itself is elastically deformed, whereby it can be retracted from a groove 136 between the connecting member 132 and 133. By utilizing this, the above handle unit 104 is fixedly assembled to be retractably engaged with the vibrator unit 102.

In addition, a small-diameter coupling cylinder portion 132b is formed at the rear end portion of the connecting member 132. A cylindrical portion 157b at the rear end side of the contact member 157 is inserted into this coupling cylinder portion 132b. Here, the protuberance 157a at the rear end of the above cylindrical portion 157b is set in a state in which its outer diameter is larger than the inner diameter of the coupling cylinder portion 132b. Then, in a state in which the cylindrical portion 157b of the contact member 157 is elastically deformed by the slit construction, the protuberance 157a comes into contact with the inside of the above connecting member 132, and in general is engaged with the peripheral groove 132a. At this time, an abnormally shaped portion is engaged with the slider receptacle member 149, whereby the contact member 157 rotates integrally in an axial periphery direction.

As shown in FIG. 25, an internal end portion of a high-frequency connecting pin 160 is mounted on the upper part of the rear end of the stationary handle 127 in a screwed manner. This high-frequency connecting pin 160 is electrically connected to the above connecting member 132. An active cable (not shown) for supplying a high-frequency current by means of a high-frequency quenching power unit (not shown) is connected to an outer end portion of the high-frequency connecting pin 160. An insulating cover 160a is covered at a portion exposed in a state in which the above active cable (not shown) is mounted on this high-frequency connecting pin 160. In this manner, electrical safety can be ensured.

In the above construction, when the rotary knob 126 is rotated, the contact member 157 and probe 103 also rotate integrally coaxially via the connecting member 144, drive force transmission intermediate member 148, and slider receptacle member 149 concurrently. In addition, during this rotating operation, the protuberance 157a of the contact member 157 is always brought into contact with the connecting member 132 with elasticity force of the contact member 157, and is electrically connected thereto. In this manner, the high-frequency connecting pin 160 and probe 103 are electrically connected to each other via the connecting member 132, contact member 157, and connecting rubber 158. Thus, via these elements, a high-frequency current is supplied to the distal end portion of the probe 103, and high-frequency treatment with an organism tissue can be carried out with such a high-frequency current.

In addition, as shown in FIG. 25 and FIG. 26, an operating pin 134 is mounted on the movable handle 129. This operating pin 134 is engaged with a recess 141a consisting of a peripheral groove of a slider member 141 in the stationary handle 127.

Then, when the movable handle 129 is turned, the operating pin 134 moves in an axial direction together with such turning. Together with movement of this operating pin 134, the slider member 141 existing in the stationary handle 127 moves in an axial direction in the range equal to or smaller than a quantity of equipment load of the spring 151, thereby transmitting a drive force. By this drive force, a distal end acting portion 115 described later is designed to be turned.

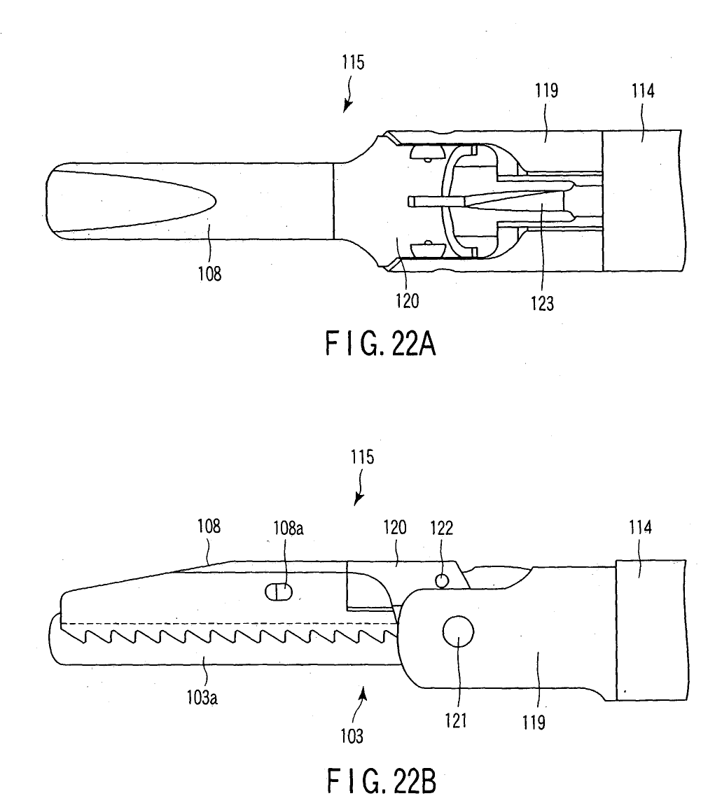

Now, the distal end acting portion 115 provided at a distal end of the insert portion 114 will be described with reference to FIG. 22A and FIG. 22B. The distal end acting portion 115 has a seizing portion 108 and a jaw 120. The seizing portion 108 is attacked to the jaw 120 in a locked state, as described later. The seizing portion 108 is disposed at a position opposed to a handling portion 103a consisting of the distal end portion of the probe 103, whereby an organism tissue can be seized between the handling portion 103a of the probe 103 and the seizing portion 108 thereof.

A distal end cover 119 is fixed at the distal end of the above outer pipe 117. The above distal end acting portion 115 is assembled with the distal end cover 119 thereof. An electrically insulation retaining member 139 consisting of a material with a low frictional coefficient formed in a cylindrical shape is arranged inside of this distal end cover 119. By this retaining member 139, the distal end cover 119 is prevented from coming into direct contact with the above probe 103.

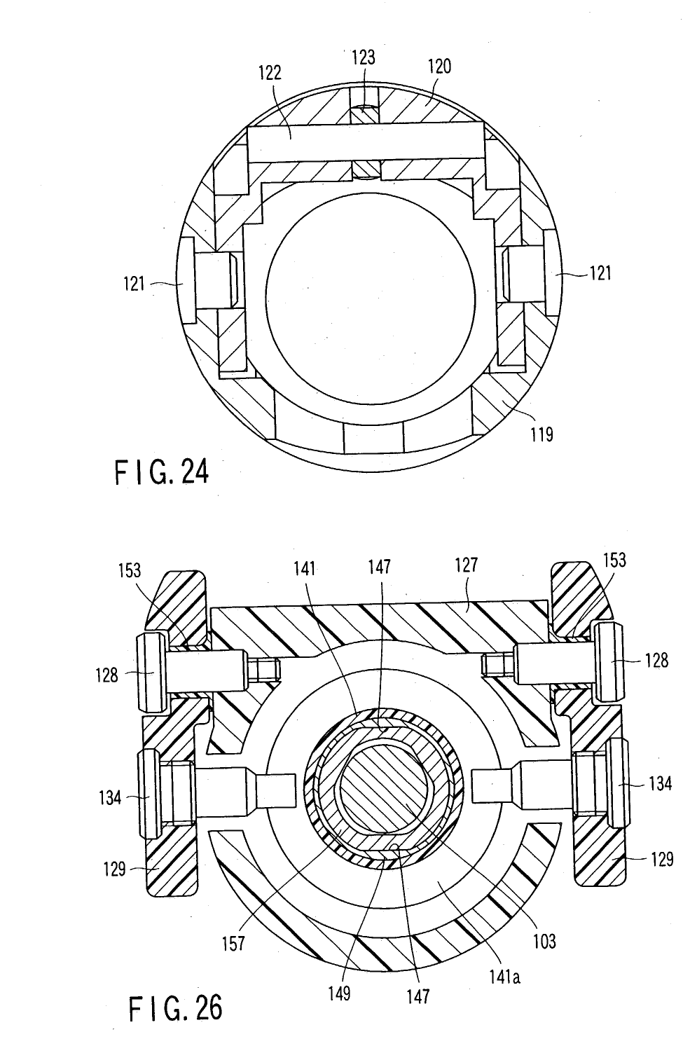

The jaw 120 of the distal end acting portion 115 is turnably mounted on the distal end cover 119 by two supporting pins 121 disposed at the right and left of the frontal end of the distal end cover 119. A distal end of the above drive shaft 123 is coupled with the rear end portion of the jaw 120 (refer to FIG. 24). At the rear end side, this drive shaft 123 passes between the distal end cover 119 and the outer pipe 117 and inner pipe 118 in the insert portion 114, is extended to an operating portion 116, and is coupled with the slider receptacle member 149. Then, the drive shaft 123 is retracted by means of the slider member 141 which is moved by means of the movable handle 129, thereby turning the above jaw 120.

In addition, at the jaw 120, a pair of top and bottom protuberances 120b are provided so as to be forwardly protruded. Between the top and bottom protuberances 120b, an elastically deformable slit 120a is formed with a predetermined quantity of force. A protuberance 120c bulged to the outside is provided at the distal end portion of this protuberance 120b. Elastic action is imparted to this protuberance 120b by means of the slit 120a. Then, the protuberance 120c is designed to be engaged with a seizing portion 108 described later in a snap lock manner. The seizing portion 108 is removably connected to the above jaw 120.

Now, a construction of a connection portion for removably connecting the seizing portion 108 to the jaw 120 will be described with reference to FIG. 23. The seizing portion 108 has a mount member 124 and a seizing member 142. The seizing member 142 is formed so as to cover the periphery of the mount member 124.

An engaging hole 124a is formed at the rear end portion of the above mount member 124. The protuberance 120b of the above jaw 120 is designed to be removably inserted into this engaging hole 124a.

A lock pin 161, a coil shaped spring 162, and a fixing pipe 163 are provided in the engaging hole 124a. The lock pin 161 is movably mounted in an axial direction on the depth side of the engaging hole 124a. The coil shaped spring 162 is wound at the outer periphery of the lock pin 161. The fixing pipe 163 is fixed to be intimately engaged with an inlet side of the engaging hole 124a.

In addition, a flange 161a is formed at the outer periphery of the axial proximal end side of the lock pin 161. A head portion 161b is formed at the proximal end portion more than this flange 161a. The spring 162 is intervened to be compressed between the internal end wall of the engaging hole 124a and the flange 161a of the lock pin 161. By means of this spring 162, the lock pin 161 is biased toward the fixing pipe 163.

FIG. 23 shows an mounted state in which the seizing portion 108 is assembled with the jaw 120. In this mounted state, a head portion 161b of the lock pin 161 enters the distal end portion of a slip 120a of the protuberance 120b of the jaw 120, and at the same time, the flange 161a abuts against a distal end of the protuberance 120b. In addition, a projecting portion 120c of the protuberance 120b of the above jaw 120 is engagingly locked at an inward end of the fixing pipe 163.

Namely, the seizing portion 108 is connected to the jaw 120 in a locked state in a snap lock manner. In this case, the protuberance 20a having a slit 120a is engaged into the fixing pipe 163. Further, the projecting portion (engagingly lock portion) 120c of the protuberance 120b is engaged with an inward end (engagingly lock portion) of the fixing pipe 163. Further, the lock pin 161 is biased in the axial proximal end direction by means of the spring 162. Thus, the head portion 161b of the lock pin 161 enters the slit 120a, and is engaged with the protuberance 120b. Then, a mechanism for suppressing elastic deformation to the inside of the protuberance 120b is configured. Therefore, the jaw 120 is automatically attached to the seizing portion 108 by means of engaging operation. Once the jaw is temporarily attached, it is not easily removed. In an assembled state in which the jaw is fixedly attached in a locked form in this manner, the seizing portion 108 is not easily removed even if it is pulled in its pullout direction.

Further, as shown in FIG. 28, a hole 108a is formed on the side face of the seizing portion 108. In the present embodiment, a hole 108a penetrates so as to laterally cross the seizing portion 108. An unlocking pin 166 of a disassembling jig 164 described later is designed to be inserted into this hole 108a.

Now, the above disassembling jig 164 will be described here. The disassembling jig 164 has a positioning face 165a formed in a latch (substantial L shape), as shown in FIG. 28. The distal end acting portion 115 of the ultrasonic operating apparatus 101 abuts against this positioning face 165a, and is set in a bonded state. At this time, the distal end acting portion 115 is set in a state in which the seizing portion 108 is mounted on the jaw 120. Then, this acting portion is bonded in abutment against the positioning face 165a of the disassembling jig 164 from the side face of the distal end acting portion 115 over the distal end face.

On the side face of this positioning face 165a, the above unlocking pin 166 is protruded in a vertically abutting state. The unlocking pin 166 is formed to be in diameter which is smaller than a width of the slit 120a in the protuberance 120b of the jaw 120 and which does not inhibit compressive deformation of the protuberance 120b.

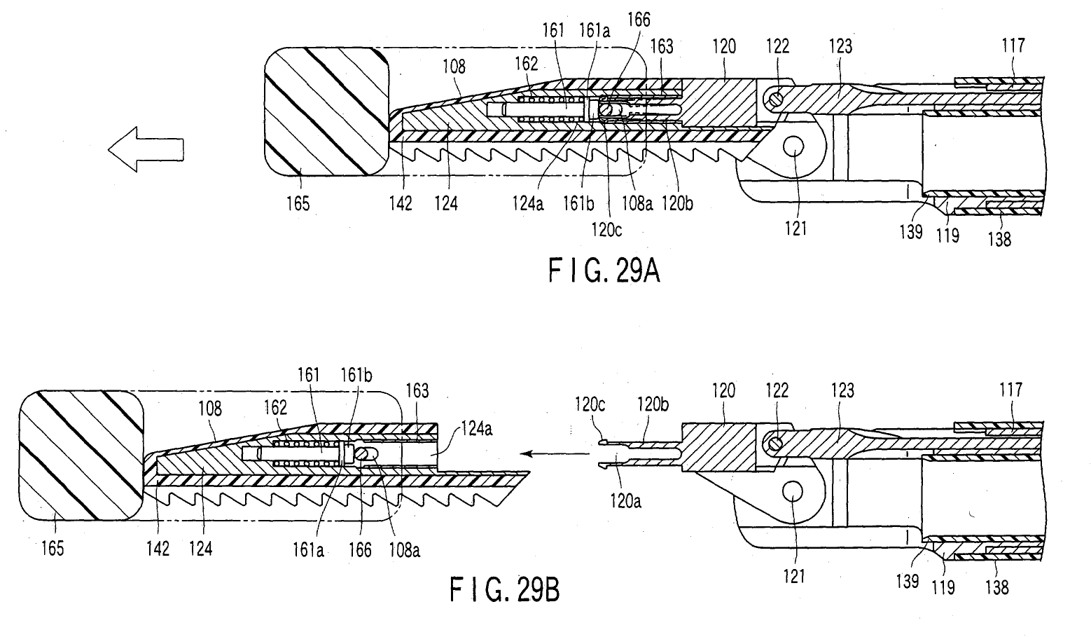

Now, a work of removing the seizing portion 108 from the jaw 120 by using the above disassembling jig 164 will be described here. First, as shown in FIG. 28, the disassembling jig 164 is set so as to abut the positioning 165a from the side face over the distal end face of the distal end acting portion 115 having the seizing portion 108 mounted thereon. At this time, the unlocking pin 166 naturally enters the hole 108a of the seizing portion 108. In addition, when the unlocking pin 166 is inserted into the hole 108a of the seizing portion 108, the unlocking pin 166 naturally enters the slit 120a, as shown in FIG. 29A.

In this state, the disassembling jig 164 is operated to be pulled in a direction indicated by the arrow in FIG. 28. By this operation, as shown in FIG. 29A, the unlocking pin 166 abuts against the head portion 161b of the lock pin 161. Then, when pullout operation is carried out until the head portion 161b of the lock pin 161 slips off from the slit 120a in a direction indicated by the arrow in FIG. 29A, the protuberance 120b of the jaw 120 is released from the lock pin 161. That is, the lock action of the lock pin 161 is released, and thus, the protuberance 120b of the jaw 120 can be deformed, and a locked state is released. Then, by pulling the seizing portion 108 in a direction indicated by the arrow, as shown in FIG. 29B, the seizing portion 108 can be removed from the jaw 120.

On the other hand, in the case where the seizing portion 108 is mounted on the jaw 120, the protuberance 120b of the jaw 120 may be inserted into the engaging hole 124a of the seizing portion 108 from the distal end side. Thus, a work of mounting the seizing potion 108 on the jaw 120 can be carried out without using the disassembling jig 164. At this time, the protuberance 120b can be deformed, and thus, enters the engaging hole 124a so that the lock pin 161 can be pushed more forwardly than the inner end position of the fixing pipe 163. The deformation of the protuberance 120b is restored at a time when the projecting portion 120c of the protuberance 120b is forwarded more than the inner end position of the fixing pipe 163. In this manner, the projecting portion 120c is engagingly locked with the inner end of the fixing pipe 163. The lock pin 161 is biased by means of the spring 162. Thus, as shown in FIG. 23, the head portion 161b of the lock pin 161 enters the distal end portion of the slit 120a at the protuberance 120b of the jaw 120, and the flange 161a of the lock pin 161 is automatically restored to a position abutting against the distal end of the protuberance 120b. Then, the protuberance 120b in the above jaw 120 expands, and the projecting portion 120c is engagingly locked with the inner end of the fixing pipe 163. In this manner, the seizing portion 108 can be easily mounted so as to be automatically locked with the jaw 120. In the case where the seizing portion 108 is thus mounted on the jaw 120, it can be easily mounted without using the disassembling jig 164.

As described above, in the present embodiment, in the case where the seizing portion 108 is removed from the jaw 120, the dedicated disassembling jig 164 is used. However, when the seizing portion 108 is mounted on the jaw 120, it can be easily mounted without the dedicated disassembling jig 164.

Lastly, a description will be given with respect to an operating method for treating an organism tissue in a ultrasonic manner by using the above constructed ultrasonic operating apparatus 101. A finger is hooked with the stationary handle 127 and movable handle 129 of the handle unit 104, and the movable handle 129 is turned. At this time, an operating pin 134 coupled with the movable handle 129 turns via the supporting pin 128, and the slider member 141 engaged with the operating pin 134 advances or retracts in an axial direction according to the turning orientation. Further, the drive force transmission intermediate member 148 coupled with the slider member 141 via the spring 151 advances and retracts, and the drive shaft 123 advances or retracts via the drive force transmitting pin 140. In this manner the jaw 120 at the distal end of the insert portion turns via the supporting pin 121.

By this operation, when an organism tissue is sandwiched between the seizing member 142 of the distal end acting portion 115 and the probe 103, the seizing member 142 turns at a predetermined angle with the pin 121 being a fulcrum in accordance with deflection of the probe 103 so that a force is uniformly applied over the full length of the seizing portion 108. In this state, when a ultrasonic wave is outputted to the probe 103, coagulation or dissection of an organism tissue such as blood vessel can be carried out.

In addition, a high-frequency current is supplied from a high-frequency connecting pin 160 to the distal end portion of the probe 103, thereby making it possible to carry out high-frequency treatment of an organism tissue by using a high-frequency current.

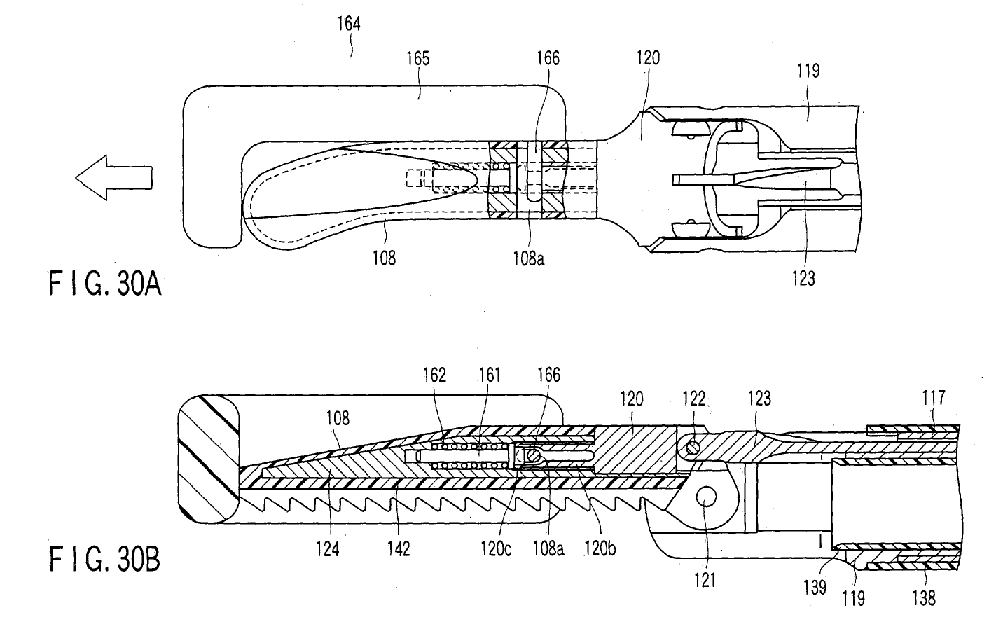

The seizing portion 108 of the ultrasonic operating apparatus 101 of the present embodiment can be applied to a seizing portion whose distal end portion is formed in a curved shape, as shown in FIG. 30.

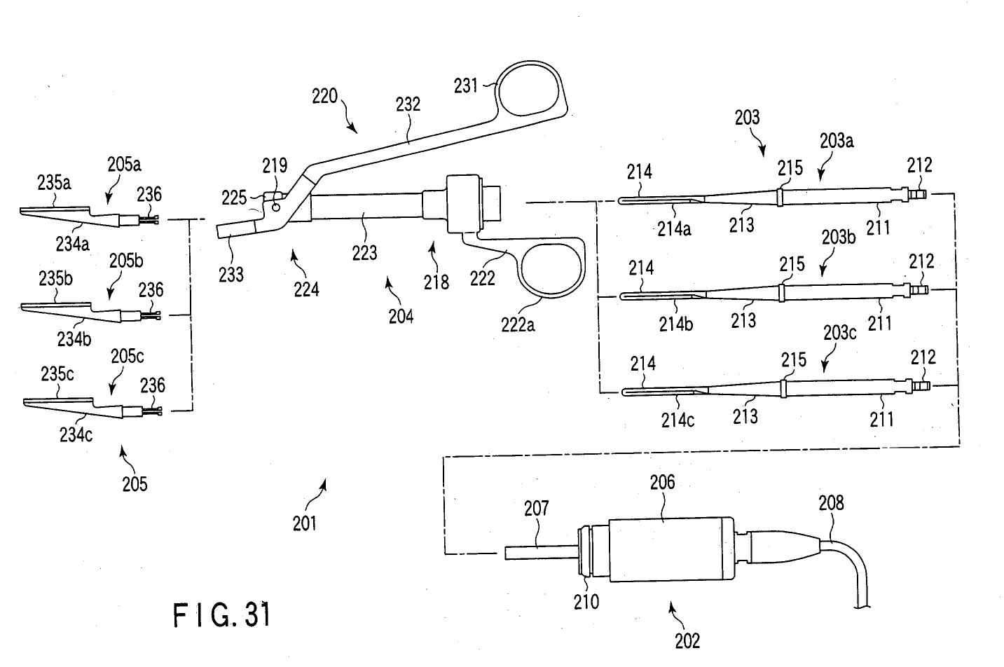

In addition, FIG. 31 to FIG. 36D show a fourth embodiment of the present invention. FIG. 31 shows a schematic configuration of an entire system of a ultrasonic operating apparatus 201 according to the present embodiment. The system of this ultrasonic operating apparatus has one vibrator unit 202, a probe unit group 203, one handle unit 204, and an operating unit group 205. The probe unit group 203 has one probe unit 203a, one or a plurality of probe unit replacing members 203b and 203c, two of which are in the present embodiment. The operating unit group 205 has one operating unit 205a and one or a plurality of operating unit replacing members 205b and 205c, two of which are in the present embodiment.

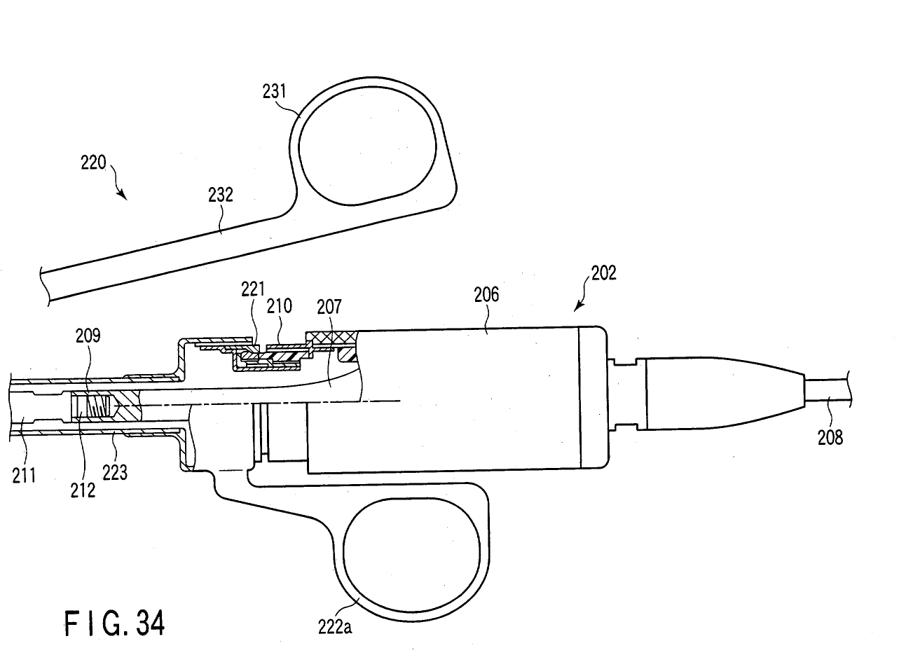

A cylindrical cover 206 is provided at the vibrator unit 202, as shown in FIG. 34. In this cover 206, there are provided: a ultrasonic vibrator (not shown) for generating ultrasonic vibration; and a horn 207 for amplifying ultrasonic vibration.

Further, one end of a hand piece cable 208 is coupled with a proximal end portion of the vibrator unit 202. The other end of this hand piece cable 208 is electrically connected to a power unit (not shown). Then, power is supplied from the power unit to the ultrasonic vibrator, whereby the ultrasonic vibrator is vibrated.

In addition, a proximal end portion of the horn 207 is connected to the ultrasonic vibrator. Then, the ultrasonic vibration generated by means of the ultrasonic vibrator is amplified by means of this horn 207, and the amplitude is expanded up to a first stage. Further, a screw hole portion 209 is formed at a distal end of the horn 207. A proximal end of the probe unit 203a is mounted on this screw hole portion 209. A coupling portion 210 for coupling with the handle unit 204 is provided at a distal end portion of the vibrator unit 202.

The probe unit 203a is formed as a rod shaped vibration transmitting member 211 for transmitting the ultrasonic vibration generated by means of the ultrasonic vibrator. A male screw portion 212 is formed at a proximal end of this vibration transmitting member 211. This male screw portion 212 is screwed into a screw hole portion 209 of the horn 207.

Further, as shown in FIG. 31, a second horn 213 and a distal end allowance portion 214a positioned at the distal end side of this second horn 213 are provided at the vibration transmitting member 211. The second horn 213 further expands the amplitude of the ultrasonic vibration amplified by means of the horn 207 up to a second stage.

The ultrasonic vibration from the ultrasonic vibrator, the vibration amplified by means of the horn 207 of the vibrator unit 202 and the second horn 213 of the vibration transmitting member 211, is transmitted to the distal end allowance portion 214a, whereby the distal end allowance portion 214a vibrates.

In addition, in the present embodiment, a length of the probe unit 203a is set in a length corresponding to a 1/2 wavelength of ultrasonic vibration. At the intermediate portion in the axial direction of this vibration transmitting member 211, a substantially ring shaped protuberance 215 is formed to be protruded in a diametrical direction. This protuberance 215 is disposed at a position of a node of the ultrasonic vibration transmitted by means of the vibration transmitting member 211.

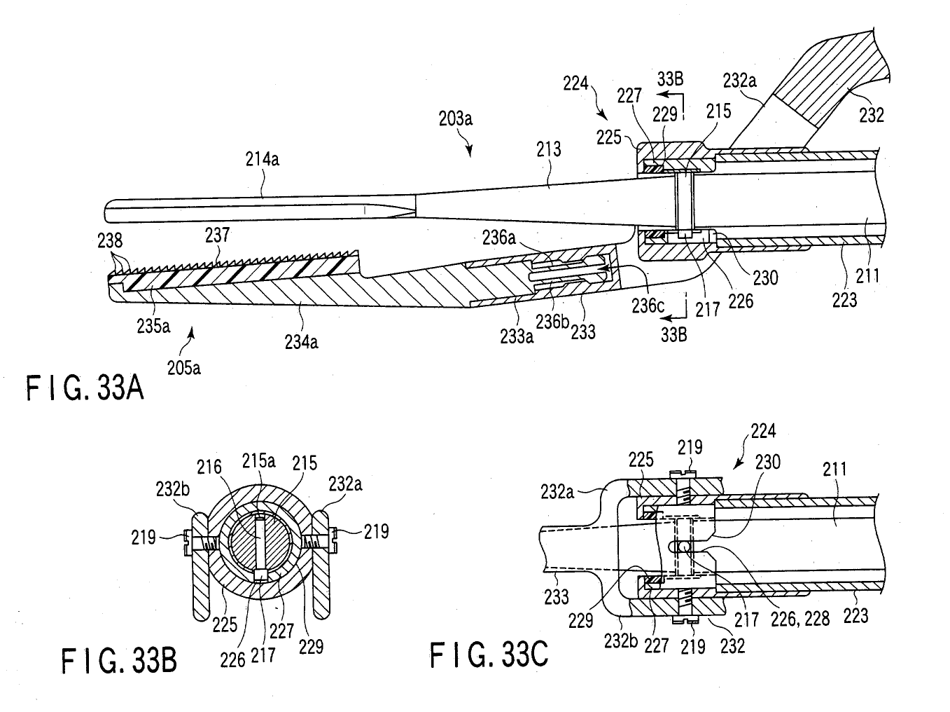

As shown in FIG. 33B, a pin through hole 215a penetrating in a diametrical direction is formed at this protuberance 215. A rotation restricting pin 216 is inserted into this pin through hole 215a, and is integrally fixed thereto. Here, a cylindrically shaped head portion 217 which is larger in diameter than the pin through hole 215a is formed at one end of the rotation restricting pin 216.

In addition, one probe unit 203a with the above described construction and one or a plurality of probe unit replacing members 203b and 203c, two of which are in the present embodiment, are provided for the probe unit group 203. Here, the distal end allowance portions 214b and 214c which are different from the distal end allowance portion 214a of the probe unit 203a in length and shape are provided at the two probe unit replacing members 203a and 203c. For example, a length of the distal end allowance portion 214b of the first probe unit replacing unit replacing member 203b on one hand is set to be shorter than that of the distal end allowance portion 214a of the probe unit 203a. A length of the distal end allowance portion 214c of the second probe unit replacing member 203bc on the other hand is set to be further shorter than that of the distal end allowance portion 214b of the first probe unit replacing member 203b. With respect to the two probe unit replacing members 203b and 203c, portions other than the distal end allowance portions 214b and 215c are constructed to be substantially identical to the probe unit 203a, respectively.

In addition, a first handle 218 at the fixing portion side and a second handle 220 are provided at the handle unit 204. This second handle 220 is turnably mounted on the first handle 218 via a handle pivoting shaft 219.

An elongated pipe 223 into which the probe unit 203a is to be inserted is provided at the first handle 218. A vibrator connecting portion 221 is provided at a proximal end portion of this pipe member 223. A coupling portion 210 of the vibrator unit 202 is removably connected to this vibrator connecting portion 221.

Further, a backward extension portion 222 extended backwardly is formed at the outer periphery side of the vibrator connecting portion 221. A finger insertion ring 222a is provided at a terminal portion of this rear expansion portion 222.

In addition, a support portion 224 for supporting the probe unit 203a and the second handle 220 is provided at the distal end side of the pipe member 223 of the first handle 218. The position of this support portion 224 is set in a state in which the support portion is disposed at a position matched to that of the protuberance 215 of the probe unit 203a during a work of assembling the vibrator unit 202, probe unit 203, handle unit 204, and operating unit 205a.

Further, at the support portion 224, a circular second handle support member 225 disposed at a distal end portion of the pipe member 223 is provided as shown in FIG. 33A. On the inner periphery face of the distal end portion of this second handle support member 225, a ring shaped outside support member 227 and an inside support member 229 are engagingly fitted as shown in FIG. 33B. Here, the outside support member 227 is formed of a hard member such as stainless or PEEK. At this outside support member 227, a slit 226 engaged with the head portion 217 of the rotation restricting pin 216 of the probe unit 203a is formed as shown in FIG. 33C. A guide portion 230 outwardly opened is formed at a free end of this slit 226. Then, by this guide portion 230, the rotation restricting pin 216 is guided to the slit 226.

In addition, the inside support member 229 is formed of a fluorine resin such as PTFE with a small frictional coefficient. An internal diameter of this inside support member 229 is set to be substantially equal to an outer diameter of the protuberance 215 of the vibration transmitting member 211. Further, a slit 228 whose shape is identical to the slit 226 of the outside support member 227 is formed at this inside support member 229.

In addition, an elongated arm 232 is formed at the second handle 220. At a distal end portion of this arm 232, a two-way portions 232a and 232b disposed at both sides of the second handle support member 225 are provided as shown in FIG. 33B and FIG. 33C. Then, in a state in which the two-way portions 232a and 232b of this arm 232 sandwich the second handle support member 225 at both sides, the second handle 220 is turnably mounted on the second handle support member 225 via a pair of handle pivoting shaft 219.

Further, a ring 231 for insertion into a terminal portion of a proximal end portion of the arm 232 with fingers is provided at the second handle 220. In addition, a substantially rod shaped jaw connecting portion 233 to which the handling portion unit 205 is removably connected is provided at the terminal portion at the distal end side of the arm 232. A connection hole 233a is formed at a distal end portion of this jaw connecting portion 233.

In addition, at the operating unit 205a, there are provided: a jaw 234a made of a metal such as stainless; and a seizing member 235a consisting of a fluorine resin such as PTFE integrally mounted on this jaw 234a. Here, a handle connecting portion 236 for removably making connection with the jaw connecting portion 233 of the handle unit 204 is provided at the proximal end side of the jaw 234a. A snap fit type connection mechanism portion 236c is provided at the handle connecting portion 236 according to the present embodiment. This connection mechanism portion 236a has a pair of movable arms 236a and 236b which is operated to be opened and closed by elastic deformation.

In the present embodiment, although the handle connecting portion 236 is formed as the snap fit type connection mechanism portion 236c, a connection mechanism portion between the proximal end side of the jaw 234a and the jaw connecting portion 233 of the handle unit 204 may be formed by using another method such as a detaching mechanism with screws.

In addition, a seizing face 237 for seizing an organism tissue between distal end allowance portion 214a of the probe unit 203a and the seizing face is formed on the top face of the seizing member 235a. At both sides of this seizing face 237, a plurality of teeth 238 are formed along the longitudinal direction of the seizing face 237, as shown in FIG. 33A.

In addition, one operating unit 205a having the above described construction and one or more operating unit replacing members 205b and 205c, two of which are in the present embodiment, are provided for the operating unit group 205. Here, seizing members 235b and 235c in length and shape corresponding to the distal end allowance portions 214b and 214c of two probe unit replacing members 203b and 203c are formed, respectively, at the two operating unit replacing members 205b and 205c. For example, the seizing member 235b of the first operating unit replacing member 205b on one hand is set in length and shape corresponding to the distal end allowance portion 214b of the first probe unit replacing member 203b, respectively. The seizing member 235c of the second operating unit replacing member 205c on the other hand is set in length and shape corresponding to the distal end allowance portion 214a of the second probe unit replacing member 203, respectively.

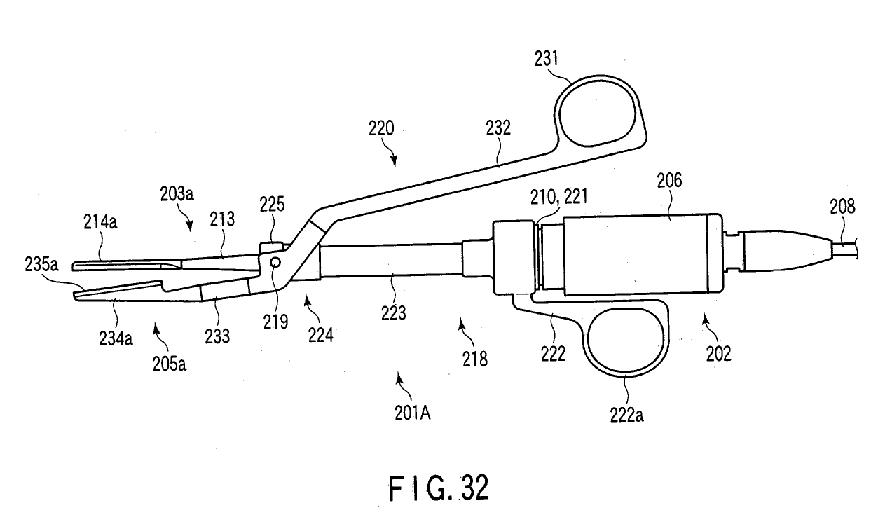

Then, in the system of the ultrasonic operating apparatus 201 according to the present embodiment, one vibrator unit 202, one probe unit 203a, one handle unit 204, and one operating unit 205a are removably assembled, respectively, and a ultrasonic operating apparatus main body 201A is formed as shown in FIG. 32.

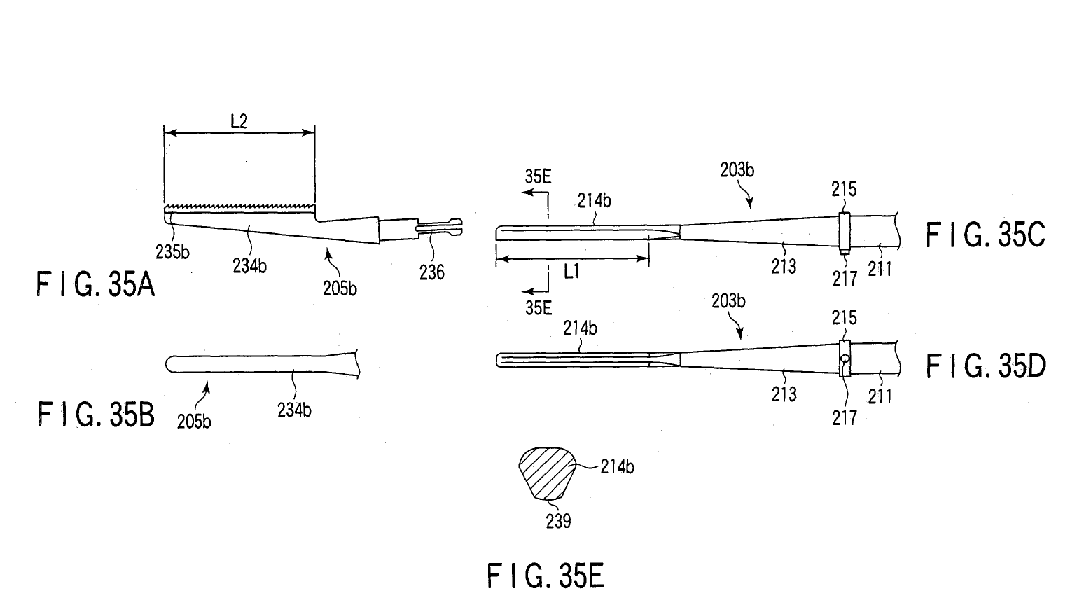

In addition, FIG. 35C and FIG. 35D show a first probe unit replacing member 203b and FIG. 35A and FIG. 35B show a first operating unit replacing member 205b., respectively. These figures each show an example of the probe unit 203a and operating unit 205a, or alternatively, a plurality of provided probe unit replacing member and operating unit replacing member.

As shown in FIG. 35C and FIG. 35D, a distal end allowance portion 214b of a length L1 extending straight in a linear shape in the axial direction of the vibration transmitting member 211 is formed at the distal end portion of the first probe unit replacing member 203b according to the present embodiment. Further, the sectional shape of the distal end allowance portion 214b is formed in a substantially triangular shape, as shown in FIG. 35E. Here, a top portion 239 of the distal end allowance portion 214b is designed to come into contact with the seizing face 237 of the seizing member 235b.

As shown in FIG. 35A and FIG. 35B, a jaw 234b and a seizing member 235b in length L2 extending in a straight way to act in collaboration with the distal end allowance portion 214b is provided at a distal end portion of the first operating unit replacing member 205b. Here, a length L1 of the distal end allowance portion 214b of the first probe unit replacing member 203b is set so as to be substantially equal to a length L2 of the seizing member 235b of the first operating unit replacing member 205b.

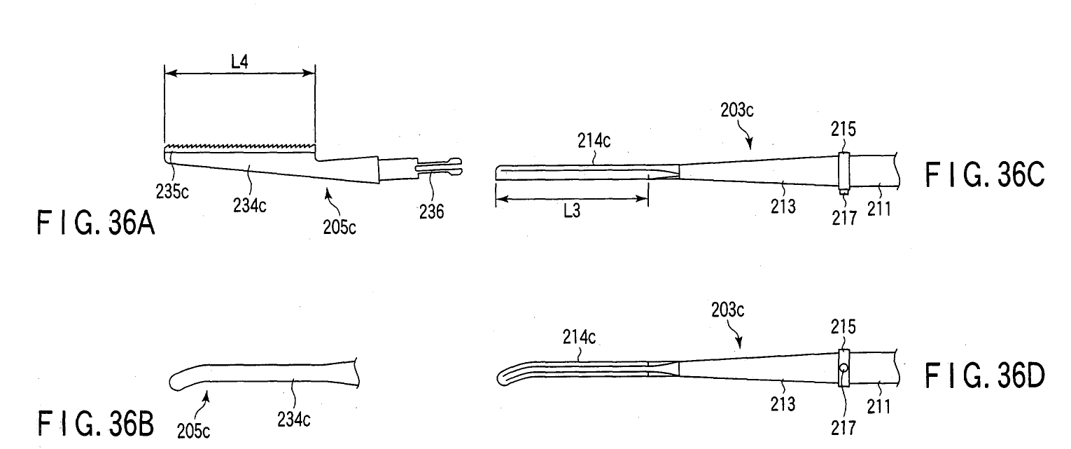

In addition, FIG. 36A and FIG. 36B show a second operating unit replacing member 205c, and FIG. 36C and FIG. 36D show a second probe unit replacing member 203, respectively. These figures each show another example of the operating unit 205a and probe unit 203a, or alternatively, a plurality of provided probe unit replacing members and operating unit replacing members.

Here, at a distal end portion of the prove unit replacing member 203c, a distal end allowance portion 214c shaped to be curved toward a distal end is formed as shown in FIG. 36D. Further, at a distal end portion of the second operating unit replacing member 205c, there are provided: a jaw 234c and a seizing member 235c of the operating unit 205a shaped to be curved toward a distal end in the same way as that in the distal end allowance portion 214c to act in collaboration with the distal end allowance portion 214c of the probe unit replacing member 203c. Here, a length L3 of the distal end allowance portion 214c of the probe unit replacing member 203 is set so as to be substantially equal to a length L4 of the seizing member 235c of the first operating unit replacing member 205c.

In addition to the first probe unit replacing member 203b and the first handling portion unit replacing member 205b shown in FIG. 35A to FIG. 35E or the second probe unit replacing member 203c and the second operating unit replacing member 205c, there may be provided: a probe unit 203a and an operating unit 205a in a variety of lengths or shapes, or alternatively, a plurality of provided probe unit replacing members and allowance portion unit replacing members.

Now, advantageous effect of the above construction will be described here. First, assembling of the ultrasonic operating apparatus 201 according to the present embodiment will be described here. When the ultrasonic operating apparatus 201 according to the present embodiment is used, a probe unit suitable for treatment of diseases or surgical operation is selected from among the probe unit group 203, or alternatively, a preferable probe unit is selected from among the two probe unit replacing members 203b and 203c. Further, a operating unit corresponding to the thus selected probe unit from among the operating unit group 205 is selected. Here, for example, the probe unit 203a and operating unit 205a are selected.

Then, a handle connecting portion 236 of the operating unit 205a is connected to a jaw connecting portion 233 of the handle unit 204. In this manner, the operating unit 205a is assembled with the handle unit 204.

Then, a male screw portion 212 of the probe unit 203a is screwed into a screw hole portion 209 of the vibrator unit 202, and the probe unit 203a is mounted on the vibrator unit 202. Subsequently, the vibrator unit 202 having the probe unit 203a mounted therein is inserted into the handle unit 204 from the rear side end. At this time, the probe unit 203a is inserted into the pipe member 223 of the handle unit 204 from the distal end side.

In addition, during a work of inserting this probe unit 203a, the head portion 217 of the rotation restricting pin 216 of the probe unit 203a is guided along a guide portion 230 of the handle unit 204, and the head portion 217 of the rotation restricting pin 216 is guided into a slit 226 and a slit 228. In this state, if the vibrator unit 202 is further pushed against the handle unit 204, a coupling portion 210 of the vibrator unit 202 is engaged with a vibrator connecting portion 221 of the handle unit 204, and the vibrator unit 202 is fixed to the handle unit 204. At this time, the slit 226 and head portion 217 are engaged with each other, whereby positioning in the rotation direction of the probe unit 203a with respect to the handle unit 204 is carried out.

Now, a work of treating an organism tissue by using the ultrasonic operating apparatus 201 will be described here. First, the first handle 218 and the second handle 220 are operated to be opened to position an organism tissue between the seizing member 235a of the operating unit 205a and a distal end allowance portion 214a of a vibration transmitting member 211.

Then, in this state, the first handle 218 and the second handle 220 are operated in a closed direction to seize an organism tissue between the seizing member 235a and the distal end allowance portion 214a. Further, while the organism tissue is seized, power is supplied from a power unit to a ultrasonic vibrator to ultrasonically vibrate the ultrasonic vibrator. This ultrasonic vibration is amplified by means of a horn 207 and a second horn 213, and is transmitted to the distal end allowance portion 214a of the vibration transmitting member 211.

At this time, although deflection occurs due to a force of the seizing member 235a applied to the vibration transmitting member 211, the vibration transmitting member 211 is supported by means of the support portion 224 at the protuberance 215, thus preventing a contact with the vibration transmitting member 211 and the inner periphery face of the pipe member 223.

In addition, the protuberance 215 is provided at a node of ultrasonic vibration, and comes into contact with an inside support member 229 consisting of a fluorine resin such as PTFE with a small friction coefficient, thus making it possible to restrain undesirable vibration such as transversal vibration. Thus, vibration energy is efficiently transmitted to the distal end allowance portion 214a of the vibration transmitting member 211. In addition, generation of a frictional heat due to the ultrasonic vibration at the protuberance 215 can be restrained.

As has been described above, ultrasonic vibration is transmitted to the distal end allowance portion 214a of the vibration transmitting member 211 without a substantial loss, and the distal end allowance portion 214a is vibrated. At this time, a seized tissue coming into contact with the distal end allowance portion 214a is dissected by means of ultrasonic vibration and is coagulated with a frictional heat.

In treating a disease, in the case where an attempt is made to use a combination between a probe unit and a handling portion unit in shape different from a combination between the probe unit 203a and operating unit 205a currently used (for example, in the case where an attempt is made to use a combination between a first probe unit replacing member 203b and a first operating unit replacing member 205b), the probe unit 203a and operating unit 205a are removed from the ultrasonic operating apparatus main body 201A. Then, the first probe unit replacing member 203b and the first operating unit replacing member 205b may be assembled with the ultrasonic operating apparatus main body 201A. In this manner, the ultrasonic operating apparatus 201 can be recombined (replaced) with the replacement apparatus of different type. In the case where a combination between the second probe unit replacing member 203a and the second operating unit replacing member 205c is used as well, they can be recombined (replaced) in the similar procedures.

In addition, in the case where the seizing member 235a of the operating unit 205a is worn and degraded by repetitive use, only the operating unit 205a can be replaced with the replacement unit.

With the above construction, the following advantageous effect is achieved. That is, in the ultrasonic operating apparatus 201 according to the present embodiment, the probe unit 203a and operating unit 205a mounted on the ultrasonic operating apparatus main body 201A are replaced with the first probe unit replacing member 203b (or second probe unit replacing member 203a) and the first operating unit replacing member 205b (or second operating unit replacing member 205c), respectively, as required, thereby making it possible to use them in a state in which the current operating apparatus is recombined with plural groups of ultrasonic operating apparatus 201. At this time, the vibrator unit 202 of the ultrasonic operating apparatus main body 201A and the handle unit 204 can be used in common in plural groups of ultrasonic operating apparatuses 201. Thus, there is provided advantageous effect that during recombination with plural types of ultrasonic operating apparatuses 201, cost reduction can be ensured by reducing the number of components in the entire system of the operating apparatus.

In addition, in the case where the seizing member 235 of the operating unit 205a is worn and degraded by repetitive use, only the operating unit 205a may be replaced with the replacement member. Thus, the durability of the entire system of the ultrasonic operating apparatus 201 can be improved, and cost reduction can be ensured at this aspect as well.

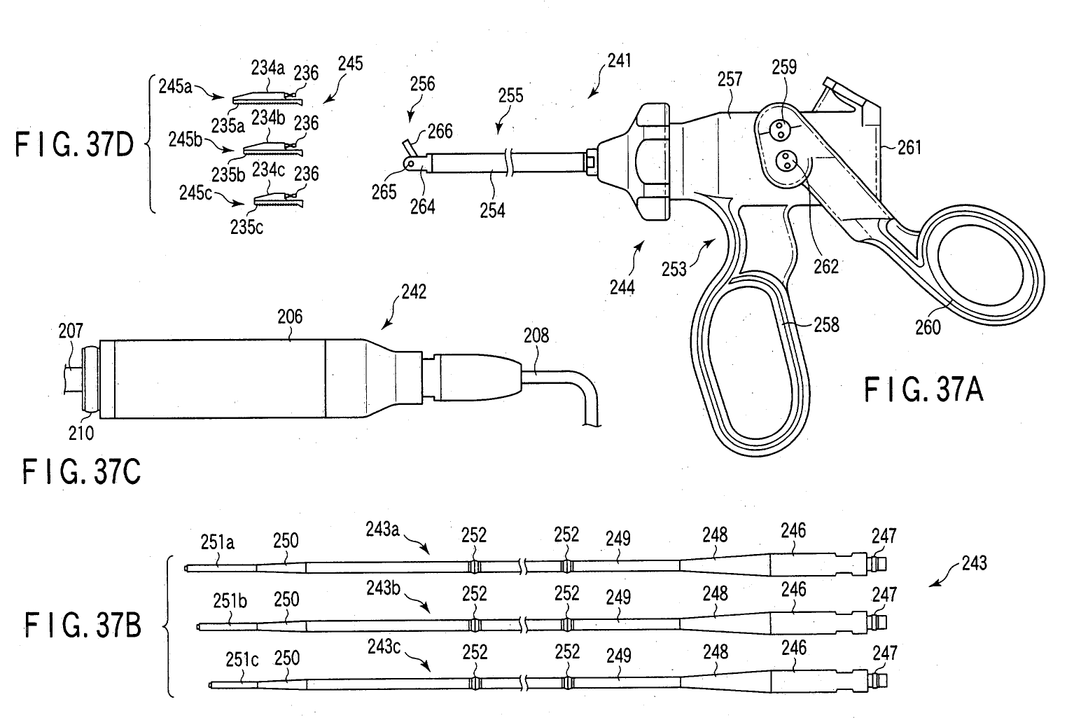

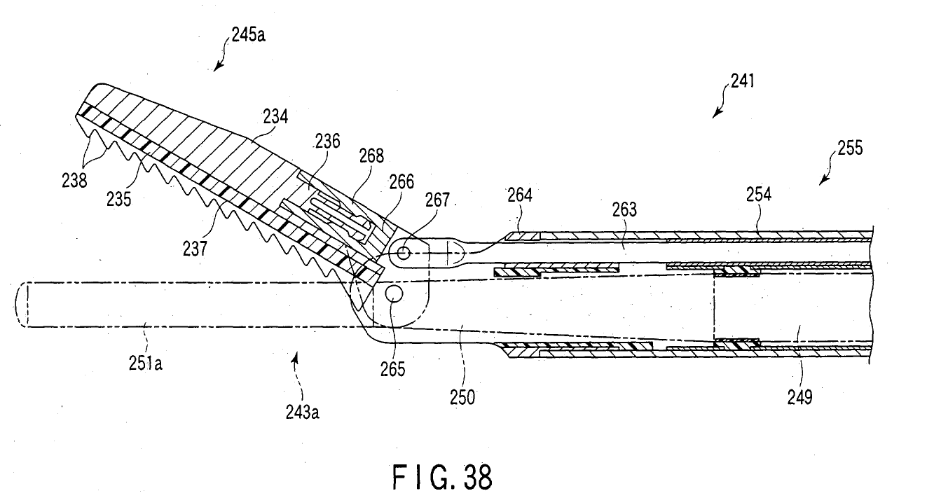

In addition, FIG. 37A to FIG. 37D and FIG. 38 show a fifth embodiment of the present invention. A system of a ultrasonic operating apparatus 241 according to the present embodiment has: one vibrator unit 242 shown in FIG. 37C; a probe unit group 243 shown in FIG. 37B; one handle unit 244 shown in FIG. 37A; and an operating unit group 245 shown in FIG. 37D. The probe unit group 243 has one probe unit 243a and one or a plurality of probe unit replacing members 243b and 243c, two of which are in the present embodiment. The operating unit group 245 has one operating unit 245 and one or a plurality of operating unit replacing members 245b and 245c, two of which are in the present embodiment.

The vibrator unit 242 is constructed in the same manner as the vibrator unit 202 according to the fourth embodiment. Here, like elements of the vibrator unit 202 according to the fourth embodiment are designated by like reference numerals. A duplicate description is omitted here.

In addition, the probe unit 243a is formed as a rod shaped vibration transmitting member 246 for transmitting the ultrasonic vibration generated by means of the ultrasonic vibrator. A male screw portion 247 screwed into a screw hole portion 209 (refer to FIG. 34) of a horn 207 is formed at a proximal end of this vibration transmitting member 246.

Further, at the vibration transmitting member 246, there are provided: a proximal end side horn 248 in which the ultrasonic vibration amplified by means of the horn 207 is further extended up to a second stage; an intermediate portion 249 positioned at the distal end side of this proximal end side horn 248; a distal end side horn 250 positioned at the distal end side of this intermediate portion 249 and extending the amplitude of the ultrasonic vibration amplified by means of the proximal end side horn 248 up to the final stage; a distal end allowance portion 251a positioned at the distal end side of the distal end side horn 250.

The ultrasonic vibration from the ultrasonic vibrator, the vibration being amplified by means of the horn 207, proximal end side horn 248, and distal end side horn 250, is transmitted to the distal end allowance portion 251a, whereby the distal end allowance portion 251a vibrates. Further, at the intermediate portion 249, a plurality of flange shaped elastic support bodies 252 are provided at a position of a node of the ultrasonic vibration transmitted by means of the vibration transmitting member 246.

In addition, one probe unit 243a having the above described construction and one or a plurality of probe unit replacing members 243b and 243c, two of which are in the present embodiment, are provided for the probe unit group 243. Here, the distal end allowance portions 251b and 251c in length and shape different from the distal end allowance portion 251a of the probe unit 243a are provided at the two probe unit replacing members 242b and 243c. For example, a length of the distal end allowance portion 251b of the first probe unit replacing member 243b on one hand is set to be smaller than that of the distal end allowance portion 251a of the probe unit 243a. A length of the distal end allowance portion 251c of the second probe unit replacing member 243 on the other hand is set to be further smaller than that of the distal end allowance portion 251b of the first probe unit replacing member 243b. Two probe unit replacing members 243b and 243c are constructed to be substantially identical to the probe unit 243a, respectively, except the distal end allowance portions 251b and 251c.

The handle unit 244 comprises: a handling portion 253; an insert sheath portion 255 consisting of an elongated covering tube 254 rotatably mounted on this handling portion 253; and a distal end acting portion 256 provided at a distal end of this insert sheath portion 255.

The operating portion 253 has a handling portion main body 257; a stationary handle 258 formed integrally with this handling portion main body 257; and a movable handle 260 turnably mounted on the operating portion main body 257 (stationary handle 258) via the handle pivoting shaft 259. A vibrator connecting portion 261 to which a vibrator unit 242 is removably connected is provided at a distal end of the operating portion main body 257.

In addition, a coupling pin 262 for coupling with a drive mechanism inside of the operating portion main body 257 is provided at the movable handle 260. The drive mechanism inside of the operating portion main body 257 is coupled with a drive shaft 263 inside of the insert sheath portion 255 shown in FIG. 38. Therefore, when the movable handle 260 is turned around the handle pivoting shaft 259, an operating force acts to the drive shaft 263, and this drive shaft 263 moves forwardly and backwardly along the axial direction.

In addition, the distal end acting portion 256 consists of: a holding member 264 mounted at a distal end portion of the covering tube 254; and a single-open type opening or closing member 266 turnably mounted on this holding member 264 via the pivoting shaft 265. As shown in FIG. 38, at the distal end side, the drive shaft 263 is coupled with a proximal end portion of the opening or closing member 266 via a pivoting pin 267. Therefore, the opening or closing of the opening or closing member 266 is carried out by operating the movable handle 260 to be opened or closed via the drive shaft 263.

In addition, a jaw connecting portion 268 to which the operating unit 245a is removably connected is provided at the distal end side of the opening or closing member 266.

The construction of the operating unit 245a is the same as that of the operating unit 205a according to the fourth embodiment. Here, like elements of the operating unit 205a according to the fourth embodiment is designated by like reference numerals. A duplicate description is omitted here.

As in the fourth embodiment, one operating unit 245a having the above described construction and one or a plurality of operating unit replacing members 245b and 245c, two of which are in the present embodiment, are provided for the operating unit group 245. Here, seizing members 235b and 235c in length and shape corresponding to the distal end allowance portions 251b and 251c of the two probe unit replacing members 243b and 243c are formed, respectively at the two operating unit replacing members 245b and 245c. For example, the seizing member 235b of the first operating unit replacing member 245b on one hand is set in length and shape corresponding to the distal end allowance portion 251b of the first probe unit replacing member 243b, respectively. The seizing member 235c of the second operating unit replacing member 245c on the other hand is set in length and shape corresponding to the distal end allowance portion 251c of the second probe unit replacing member 243c, respectively.

In a system of the ultrasonic operating apparatus 201 according to the present embodiment, one vibrator unit 242, one probe unit 243a, one handle unit 244, and one operating unit 245a are removably assembled, and the ultrasonic operating apparatus main body is formed.

Now, an operation of the above construction according to the present embodiment will be described here. Assembling of the ultrasonic operating apparatus 241 according to the present embodiment will be described here. When the ultrasonic operating apparatus 201 according to the present embodiment is used, a probe unit 243a suitable for treatment of diseases or surgical operation or either of the preferable probe units of the two probe unit replacing members 243b and 243c is selected from among the probe unit group 243. Further, an operating unit corresponding to the thus selected probe unit is selected from among the operating unit group 245. Here, for example, the probe unit 243a and operating unit 245a are selected.

Next, a handle connecting portion 236 of the operating unit 245a is engaged with a jaw connecting portion 268 of the handle unit 244. In this manner, the operating unit 245a is assembled with the handle unit 244.

Then, a male screw portion 247 of the probe unit 243a is screwed into a screw hole portion of the vibrator unit 242, and the probe unit 243a is mounted on the vibrator unit 242. Then, the vibrator unit 242 having the probe unit 243a mounted thereon is inserted into the handle unit 244 from the rear end side. At this time, the probe unit 243a is inserted into the covering tube 254 of the insert sheath portion 255 from the distal end side.

Then, when the vibrator unit 242 is further pushed against the handle unit 244, the coupling portion 210 of the vibrator unit 242 is engaged with the vibrator connecting portion 261 of the handle unit 244, and the vibrator unit 242 is fixed to the handle unit 244.

Now, a work of handling an organism tissue by using the ultrasonic operating apparatus 241 will be described here. First, the distal ends of the distal end acting portion 256 and insert sheath portion 255 to which the operating unit 245a is connected are introduced into a body cavity via a trocar punctured into the body wall.

Then, the movable handle 260 is operated in an open direction, and an organism tissue is positioned between the seizing member 235a of the operating unit 245a and the distal end allowance portion 251a of the vibration transmitting member 246. Then, in this state, the movable handle 260 is operated in a closed direction, and the organism tissue is seized between the seizing member 235a and the distal end allowance portion 251a. Further, in a state in which the organism tissue is seized, power is supplied from the power unit to the ultrasonic vibrator, and ultrasonic vibration is vibrated. This ultrasonic vibration is amplified by means of the horn 207, proximal end side horn 248, and distal end side horn 250, and is transmitted to the distal end allowance portion 251a of the vibration transmitting member 246.

In this case, the vibration transmitting member 246 is firmly held on the inner periphery face of the covering tube 254 by means of an elastic support body 252 having a plurality of outer periphery faces. Thus, undesirable vibration such as transversal vibration can be restrained, and vibration energy is efficiently transmitted to the distal end allowance portion 251a.

As has been described above, ultrasonic vibration is transmitted to the distal end allowance portion 251a of the vibration transmitting member 246 without a substantial loss, and the distal end allowance portion 251a is vibrated. At this time, the seized tissue coming into contact with the distal end allowance portion 251a is dissected by ultrasonic vibration and is coagulated with a frictional heat.

As in the fourth embodiment, in treating a disease, in the case where an attempt is made to use a combination between a probe unit and a operating unit in shape different from a combination between the probe unit 243a and the operating unit 245a currently used (for example, in the case where an attempt is made to use a combination between the first probe unit replacing member 243b and the first operating unit replacing member 245b), the probe unit 243a and operating unit 245a are removed from the ultrasonic operating apparatus main body. Then, the first probe unit replacing member 243b and the first operating unit replacing member 245b may be assembled with the ultrasonic operating apparatus main body. In this manner, the ultrasonic operating apparatus 241 can be recombined (replaced) with the replacement apparatus of different type. In the case of using a combination between the second probe unit replacing member 243c and the second operating unit replacing member 245c, recombination (replacement) can be carried out in accordance with the same procedures.

In addition, the seizing member 235a of the operating unit 245a is worn and degraded by repetitive use, only the operating unit 245a may be replaced with the replacement unit.

In the present embodiment, the probe unit 243a and operating unit 245a mounted on the ultrasonic operating apparatus main body are replaced with the first probe unit replacing member 243b (or second probe unit replacing member 243c) and the first operating unit replacing member 245b (or second operating unit replacing member 245c), respectively, as required, thereby making it possible to use plural groups of ultrasonic operating apparatuses 241 while the current apparatus is recombined with such plural groups of the apparatuses. At this time, the vibrator unit 242 of the ultrasonic operating apparatus main body and the handle unit 244 can be used in common in plural groups of ultrasonic operating apparatuses 241. Thus, as in the fourth embodiment, during recombination with plural types of ultrasonic operating apparatuses 241, there is provided advantageous effect that cost reduction can be ensured by reducing the number of components of the entire system of the operating apparatus.

Additional advantages and modifications will readily occur to those skilled in the art. Therefore, the invention in its broader aspects is not limited to the specific details and representative embodiments shown and described herein. Accordingly, various modifications may be made without departing from the spirit or scope of the general inventive concept as defined by the appended claims and their equivalents.

What is claimed is:

1. An ultrasonic operating apparatus comprising:
an elongate insert portion capable of being inserted into a body cavity;
an operating portion located on a distal end portion of the insert portion, the operating portion being used to operate an organism tissue;
a handling portion coupled to a proximal end portion of said insert portion, the handling portion having therein an ultrasonic vibrator capable of generating ultrasonic vibration;
a covering tube located around said insert portion;
a vibration transmitting member passed through the covering tube, the vibration transmitting member having an ultrasonic probe on a side of said operating portion and capable of transmitting the ultrasonic vibration from said ultrasonic vibrator to said ultrasonic probe;
a jaw rockably supported opposite said ultrasonic probe and capable of seizing the organism tissue in conjunction with said ultrasonic probe;
a control handle located in said handling portion and capable of opening and closing said jaw with respect to said ultrasonic probe; and
a handling force transmitting member coupling said jaw and said control handle, and capable of transmitting handling force from said control handle to said jaw,
said jaw including a frame-shaped jaw body having at least supporting arms arranged individually on the opposite sides of a slot extending in an axial direction of said insert portion, a tip capable of seizing the organism tissue in conjunction with said ultrasonic probe, and a joint portion removably coupling said tip between said supporting arms of said jaw body.

2. An ultrasonic operating apparatus according to claim 1, wherein said ultrasonic probe has an asymmetric curved portion curved with respect to a central axis of said insert portion.

3. An ultrasonic operating apparatus according to claim 2, wherein said curved portion is formed symmetrically with respect to a direction in which said jaw is opened or closed.

4. An ultrasonic operating apparatus according to claim 1, wherein said jaw body is designed so that support shaft portions of said tip protrude inward from the respective distal end portions of said two supporting arms, and said tip has mounting holes into which said support shaft portions are removably inserted and guide grooves for guiding said support shaft portions to said mounting holes as said tip is attached to said jaw body, said guide grooves individually having taper surfaces for movement such that the space between the respective support shaft portions of said two supporting arms widens toward said mounting holes and click step portions for preventing said support shaft portions from slipping out of said mounting holes.

5. A tool for changing a tip of an ultrasonic operating apparatus, comprising:
a tip changing tool body having an insertion hole into which a distal operating portion of said ultrasonic operating apparatus is inserted and a stopper portion for being the position of insertion of said distal operating portion inserted in the insertion hole;
a handling arm coupled to said tool body so as to be rockable around a hinge portion located on the inlet side of said insertion hole of the tool body; and
wedge-shaped separating portions adapted to be removably inserted into spaces between the tip for seizing an organism tissue and supporting arms on the opposite sides of a jaw body of said distal operating portion as the handling arm rocks, thereby moving said supporting arms in a direction such that indented fitting portions of said supporting arms and said tip are disengaged from one another.

6. A ultrasonic operating apparatus comprising:
a vibrator for generating ultrasonic vibration;
a probe removably mounted on the vibrator, the probe having an allowance portion for transmitting ultrasonic vibration from the vibrator to a distal end portion and for treating an organism tissue at the distal end portion with a ultrasonic wave; and
a distal end acting portion mounted to be detachable from the allowance portion, the distal end acting portion having a seizing portion for seizing the organism tissue between the seizing portion and the allowance portion; and
locking means for engagingly locking the seizing portion to be disengageable from the distal end acting portion, the locking means being capable of removing the seizing portion assembled with the distal end acting portion by using a dedicated tool.

7. A ultrasonic operating apparatus of claim 6, wherein the locking means has: a locking portion which utilizes elastic deformation for at least one of the distal end acting portion and the seizing portion; and a mechanism for locking both of the distal end acting portion and the seizing portion by means of the locking portion and for suppressing the elastic deformation in an locked state.

8. A ultrasonic operating apparatus of claim 6, wherein the seizing portion is released from the locked state by means of the dedicated tool.

9. A ultrasonic operating apparatus system comprising:
a vibrator unit having a ultrasonic vibrator for generating ultrasonic vibration;
a probe unit which comprises an elongated vibration transmitting member having a proximal end portion removably connected to the ultrasonic vibrator and a distal end portion at which a distal end allowance portion is arranged, the probe unit transmitting the ultrasonic vibration generated by means of the ultrasonic vibrator to the distal end allowance portion;
a handle unit removably coupled with a handling portion unit having a seizing member supported to be detachable from the distal end handling portion, the handle unit being adopted to operate the seizing member for the distal end allowance portion;
a ultrasonic operating apparatus main body with which the vibrator unit, the probe unit, and the handle unit are removably assembled;
a probe unit replacing member assembled to be replaceable with the probe unit with respect to the ultrasonic operating apparatus main body, the member having the distal end allowance portion in shape different from the probe unit; and
an operating unit replacing member assembled to be replaceable with the operating unit with respect to the ultrasonic apparatus main body, the member having the seizing member in shape corresponding to a distal end allowance portion of the probe unit replacing member, wherein corresponding parts between the probe unit replacing member and the operating unit replacing member can be selectively mounted on the ultrasonic operating apparatus main body properly.

10. A ultrasonic operating apparatus system of claim 9, wherein the handle unit has a first handle on a stationary side and a second handle turnably mounted on the first handle, the vibrator unit and the probe unit are coupled with the first handle, respectively, and the operating unit is coupled with the second handle.

11. A ultrasonic operating apparatus system of claim 9, wherein the handle unit has:
an operating portion for an operator to make operation;
an insert sheath portion having an elongated covering tube mounted on the operating portion; and
a distal end acting portion provided at a distal end of the insert sheath portion, the acting portion acting according to operation of the operating portion.
